(19) United States
(12) Patent Application Publication
(10) Pub. No.: US 2026/0275396 A1
(43) Pub. Date: Sep. 17, 2026

(54) IMPROVED METHODS OF CELL-FREE SYNTHESIS

(71) Applicant: The Board of Trustees of the Leland Stanford Junior University, Stanford, CA (US)

(72) Inventors: James R. Swartz, Menlo Park, CA (US); Max Zachary Levine, San Jose, CA (US); Medea Neek, Mountain View, CA (US)

(21) Appl. No.: 19/469,280

(22) PCT Filed: Apr. 10, 2024
(86) PCT No.: PCT/US2024/023858

§ 371 Date: Sep. 25, 2025

Related U.S. Application Data
(60) Provisional application No. 63/458,372, filed on Apr. 10, 2023.
(60) Provisional application No. 63/458,374, filed on Apr. 10, 2023.
(60) Provisional application No. 63/458,379, filed on Apr. 10, 2023.

(51) Int. Cl. C12P 19/36; C12M 1/00; C12M 1/40; C12N 11/02; C12N 11/18; C12P 7/44

(52) U.S. Cl. CPC C12P 19/36; C12M 21/18; C12M 29/06; C12N 11/02; C12N 11/18; C12P 7/44

(57) ABSTRACT

Compositions and methods are provided for improved methods of enzymatic, cell-free synthesis. The methods of the disclosure provide improvements in the manipulation of cofactors during the synthesis reactions, including NAD cofactors and Coenzyme A. In an embodiment, metabolic factors, e.g. NAD cofactors, CoA, etc. are synthesized in situ, which increases the production and/or decreases the cost of a desired biomaterial synthesized in the reaction mix. In an embodiment, reducing equivalents NADH and/or NADPH are produced from H₂. In an embodiment, aberrant NADPH tautomers are detoxified in cell-free reaction mixtures, to increase the production of a desired biomaterial.

### CROSS REFERENCE TO OTHER APPLICATIONS

This application claims the benefit of U.S. Provisional Application No. 63/458,372 filed Apr. 10, 2023, U.S. Provisional Application No. 63/458,374 filed Apr. 10, 2023, and U.S. Provisional Application No. 63/458,379 filed Apr. 10, 2023, the contents of which are hereby incorporated by reference in its entirety.

### BACKGROUND

Carbon dioxide accumulation in our atmosphere is a major cause of global warming. Our dominant fuels, biochemicals, plastics, pharmaceuticals and biomaterials all consist principally of carbon containing compounds. Therefore, tremendous advantages for the health of our planet would be gained if CO₂ from the atmosphere could be directly or indirectly incorporated into such products. However, CO₂ is a highly oxidized molecule and multiple reducing equivalents (electrons) must be supplied to convert CO₂ into such products or the precursors for producing such products. It has proven very difficult to supply these reducing equivalents to a biosynthetic process that proceeds within a living organism. There is a great need for cell-free methodology for efficiently introducing these reducing equivalents into a biosynthetic process, so that CO₂ or agriculturally produced commodities such as glucose (which contains carbon atoms derived from atmospheric CO₂) can be economically converted into biofuels, commodity biochemicals and other important products using carbon negative technologies.

Cell extracts can be activated to conduct complex metabolic processes such as protein synthesis and the inexpensive production of commodity biochemicals and other bioproducts. However, producing the cell extracts typically lowers the concentrations of metabolic cofactors and significantly lowers the rates of numerous cofactor-dependent metabolic processes. While cofactors can be added to increase rates of product formation, this adds significant expense, which could render many proposed commercial processes to be unprofitable.

A significant metabolic cofactor that is used in cell-free reactions is nicotinamide adenine dinucleotide (NAD). Reference may be made herein to nicotinamide adenine dinucleotide (NAD), nicotinamide adenine dinucleotide phosphate (NADP), and the reduced forms NADH and NADPH. In metabolism, NAD is involved in redox reactions, carrying electrons from one reaction to another. NAD and its participation in redox reactions has been harnessed by industrial applications to support the production of commodity chemicals and products in cell-free reactions. The enzymatically active form of NAD for the delivery of reducing equivalents to biosynthetic reactions is 1,4-NADPH. 1,4-NAD(P)H is consumed stoichiometrically in biocatalytic reduction reactions, but its high cost and lability present an imposing economic obstacle for industrial processes without the ability to reduce NAD(P)⁺ back to 1,4-NAD(P)H. In addition, the slow but continuous generation of non-biologically active 1,2-NAD(P)H and 1,6-NAD(P)H isomers can reduce availability of functional cofactor. While 1,2-NAD(P)H is labile, 1,6-NAD(P)H is persistent. Importantly, the 1,2- and 1,6-NAD(P)H isomers are potent inhibitors of NAD(P)H-dependent reactions. These factors are critical impediments to the direct electrochemical reduction of NAD(P)+, because the 1,2- and 1,6-NAD(P)H isomers, as well as NAD(P) dimers, are abundant side products. Inhibition by accumulated 1,6-NAD(P)H may be a cause of difficulty in metabolic engineering, and process scale-up failures.

The present disclosure relates to the production of carbon negative commodity chemicals using cell-free technology that provides precise control over metabolic fluxes, to allow for low cost, high productivity, synthesis of carbon negative commodity chemicals.

### SUMMARY OF THE INVENTION

Compositions and methods are provided for improved methods of enzymatic, cell-free synthesis. The methods of the disclosure provide improvements in the manipulation of NAD as a metabolic cofactor during the synthesis reactions. In an embodiment, metabolic factors, e.g. NAD, and/or Coenzyme A (CoA) are synthesized in situ, which increases the production and/or decreases the cost of a desired biomaterial synthesized in the reaction mix. In an embodiment, reducing equivalents NADH and/or NADPH are produced from H₂. Cell-free metabolism and a simple secondary enzymatic pathway are used to harvest reducing equivalents from H₂ and to deliver the reducing equivalents to biosynthetic pathways, in the form of NADH or NADPH. In an embodiment, aberrant NADPH tautomers are detoxified in cell-free reaction mixtures to increase the production of a desired biomaterial.

Each of the embodiments of the disclosure may be performed separately, or one or more may be combined in series, or in parallel. For example, in situ synthesized cofactors may take advantage of the H₂ process for generating reducing equivalent. Detoxification processes can be combined with one or both of in situ generation of cofactors; and generation of reducing equivalents with H₂.

The cell-free reaction may be any reaction that produces a desired product, usually reactions that involve NAD dependent reactions, including without limitation, commodity chemicals, for example succinate, proteins, small molecules, etc. In some embodiments, the methods of the disclosure increase the production of a desired biomaterial, e.g. a commodity chemical, in a cell-free reaction system. The production of a desired biomaterial may be increased by at least 10%, at least 20%, at least 30%, at least 40%, at least 50%, at least 60%, at least 70%, at least 80%, at least 90%, at least 100% or more than 100% relative to a cell-free reaction in the absence of the manipulation.

In some embodiments, synthesis is performed in a bioreactor process for the production of the desired biomaterials from readily available substrates, e.g. glucose, etc. The methods herein may utilize an integrated system that improves upon existing technologies by allowing for low cost and high productivity synthesis of, for example, carbon negative commodity chemicals that scale efficiently, using cell extracts comprising necessary enzymes in the presence of glucose, CO₂, and H₂ for the production of a desired biomaterial in a cell-free reaction mixture. The bioreactor fluid can be passed through an ultrafiltration device that is able to separate the enzymes necessary for production from the biomaterial produced. The enzymes and substrates can be returned to the reactor, while the biomaterial is directed to a capture device.

The cell-free reaction may be done at any suitable temperature. For example, the temperature may be about 15° C., about 20° C., about 25° C., about 30° C., about 35° C., about 40° C., about 45° C., or greater than 45° C. In a preferred embodiment, the temperature is from about 20-35° C.

In embodiments utilizing enzymes, e.g. for detoxification or cofactor synthesis, the cell-free reaction may be performed in a bioreactor, where the aqueous bioreactor fluid is contacted with one or a cocktail of enzymes. In some embodiments the enzymes are immobilized on a substrate, e.g. resins, particles, beads, filters, bioreactor surfaces, etc. In an embodiment, cofactor biosynthetic enzymes, which may be immobilized on a substrate, are added to a main bioreactor chamber. Any solid substrate that allows for the immobilization of the enzymes without impairing function can be used, and may use a tag for affinity attachment. For example, the substrate may include, without limitation, NiNTA beads, beads comprising antibodies or fragments thereof that specifically bind to the cofactor biosynthetic enzymes or a tag found thereon, beads comprising protein A, streptavidin beads, etc. Alternative linkage may include, without limitation, cyanogen bromide-activated (CNBr) sepharose, glutaraldehyde crosslinked onto aminopropyl-containing silica, activated organic resins, click chemistry conjugation to modified resins etc. If a resin is used as a substrate, the resin may be any size deemed necessary. For instance, the resin may be in the diameter range of 30-200 μm to ensure acceptable flow properties.

In some embodiments, a bioreactor system disclosed herein is utilized in the synthesis of biomaterials with synthetic processes requiring NADH or NADPH. In some embodiments the biomaterials are commodity biomaterials. In some embodiments, the main bioreactor chamber comprising the NAD reducing pathway enzymes also comprises the enzymes and cofactors required for synthesis of the biomaterials. In some embodiments, the main bioreactor chamber comprises active oxidative phosphorylation enzymes, e.g. to generate a supply of ATP. The synthesized biomaterial product may be continuously separated from the bioreactor fluid. The bioreactor may be fed carbohydrates continuously or in batch to provide a source of energy, and carbon as needed.

In one embodiment of the disclosure, methods and compositions are provided for the in situ synthesis of metabolic cofactors in cell-free reaction mixtures. The in situ cofactor synthesis is accomplished by providing suitable enzymes, i.e. cofactor biosynthesis enzymes; and substrates, i.e. cofactor biosynthesis substrates, in the reaction mixture to generate one or more cofactors of interest. The reaction mixture can concomitantly produce the desired biomaterial, e.g. a commodity chemical. Cofactors of interest include, without limitation, NAD; coenzyme A; thiamine pyrophosphate; vitamin B₂ (riboflavin); vitamin B₃ (niacin); vitamin B₆ (pyridoxine); vitamin B₁₂ (cyanocobalamin); biotin; folic acid; pantothenic acid; ascorbic acid; etc. In some embodiments the cofactor is NAD.

Cofactor biosynthetic enzymes can be provided in the form of a cell lysate, e.g. a cell engineered to express levels of cofactor biosynthesis enzymes suitable for producing cofactors of interest. Alternatively, lysates for production of a desired biomaterial can be supplemented with exogenous cofactor biosynthesis enzymes, which may be in solution or immobilized on a substrate.

In embodiments where synthesis of NAD as a cofactor is desired, a cell free reaction mix comprises an effective amount of enzymes including: (i) a ribokinase; (ii) a phosphoribosyl pyrophosphate (PRPP) synthetase (PRS; also known as ribose-phosphate diphosphokinase); (iii) nicotinamide phosphoribosyltransferase (NAMPT); and (iv) a nicotinamide-nucleotide adenylyltransferase (NMNAT) and substrates ribose, adenosine triphosphate (ATP) and nicotinamide. These enzymes may be referenced to as NAD biosynthesis enzymes and the substrates as NAD biosynthesis substrates. The NAD biosynthesis enzymes in the presence of ribose, ATP and nicotinamide catalyze the production of NAD⁺ where 1 ribose+1 nicotinamide+3 ATP results in the production of 1 NAD⁺+adenosine diphosphate (ADP)+adenosine monophosphate (AMP)+2 pyrophosphate (PPi). This NAD biosynthesis reaction prolongs the production of the products synthesized by cell-free reactions, and increases cell-free productivity.

NAD biosynthetic enzymes of the present disclosure can be any enzyme that performs the necessary steps to lead to the production of NAD⁺. For instance, the ribokinase may be any enzyme that falls under the enzyme commission number of EC 2.7.1.15. PRS may be any enzyme that falls under the enzyme commission number of EC 2.7.6.1. NAMPT may be any enzyme that falls under the enzyme commission number of EC 2.4.2.12. NMNAT may be any enzyme that falls under the enzyme commission number of EC 2.7.7.1. The NAD biosynthesis enzymes may also be variants of natural enzymes that are modified to enhance enzymatic activity. A biosynthetic enzyme may comprise a tag used for the detection or immobilization to a solid support, including, without limitation, polyhistidine or a variant thereof, FLAG, glutathione S-transferase (GST), green florescent protein (GFP) or a variant thereof, biotin, hemagglutinin (HA), maltose binding protein (MBP), Myc, small ubiquitin like modifier (SUMO), ubiquitin, etc.

In an embodiment, CoEnzyme A (CoA) is synthesized in situ, optionally in combination with NAD. In embodiments where synthesis of CoA as a cofactor is desired, a cell free reaction mix comprises an effective amount of enzymes including: (i) pantothenate kinase (EC 2.7.1.33); (ii) phosphopantetheinyl-cysteine synthetase (EC 6.3.2.5); (iii) 4-phosphopantetheinyl-cysteine decarboxylase (EC 4.1.1.36); (iv) pantetheine-phosphate adenylyltransferase (EC 2.7.7.3) and (v) dephospho-CoA kinase (EC 2.7.1.24) and substrates pantothenate, cysteine, CTP and ATP. These enzymes may be referenced to as CoA biosynthesis enzymes and the substrates as CoA biosynthesis substrates. The CoA biosynthesis enzymes in the presence of pantothenate, cysteine, CTP and ATP catalyze the production of CoA where 1 pantothenate+1 cysteine+CTP+3 ATP results in the production of 1 CoA+CO₂+CMP+2PPi+2ADP. This CoA biosynthesis reaction prolongs the production of the products synthesized by cell-free reactions, and increases cell-free productivity.

In one embodiment, devices and methods are provided for the detoxication of aberrant NADPH tautomers in cell-free reaction mixtures. Biologically active 1,4-NADPH can spontaneously generate non-biologically active forms of NADPH in the forms of the tautomers: 1,2-NADPH or 1,6-NADPH. Not only are these biologically inactive forms unable to be used in NADPH dependent reactions, but they also may act as inhibitors of metabolic transfer of reducing equivalents. Such catalytic rate reductions lower the productivity of cell-free reaction processes that benefit from oxidative phosphorylation as well as the cell-free production of biomaterials when the biosynthetic pathway depends upon the removal and/or addition of reducing equivalents using NAD or NADP as the reducing equivalent carriers (i.e. succinate production).

The provided methods for detoxification comprise contacting cell-free reaction fluid with one or a cocktail of NADPH detoxifying enzymes. The NADPH detoxifying enzymes selectively oxidize 1,2-NADPH and 1,6-NADPH, but not 1,4-NADPH, resulting in the production of NADP⁺. This NAD detoxification reaction prolongs the production of the products synthesized by cell-free reactions, and increases cell-free productivity.

In some embodiments the cell-free reaction is performed in a bioreactor, and the aqueous bioreactor fluid is contacted with one or a cocktail of NADPH detoxifying enzymes. In some embodiments the NADPH detoxifying enzymes are immobilized on a substrate, e.g. resins, particles, beads, filters, bioreactor surfaces, etc. In an embodiment, NADPH detoxifying enzymes, which may be immobilized on a substrate, are added to a main bioreactor chamber. In an alternative embodiment, NADPH detoxifying enzymes, which are immobilized on a substrate, are provided in an NADPH detoxification device that is operably linked to a bioreactor, where aqueous bioreactor fluid is passed over the detoxifying enzymes before being returned to the main bioreactor chamber. A stream from the bioreactor can be directed to an NADPH detoxification device, then returned to the bioreactor.

NADPH detoxifying enzymes of the present disclosure can be any enzyme that selectively oxidizes 1,2-NADPH and 1,6-NADPH but not 1,4-NADPH. NADPH detoxifying enzymes can include without limitation, pyridoxamine-phosphate oxidases (PNPOs), e.g. Saccharomyces cerevisiae Pdx3, Arabidopsis thaliana PPOX1, Escherichia coli PdxH, or Mus musculus PNPO; PNPO-related proteins (PNPO-RPs), e.g. A. thaliana PPOX1, Nostoc punctiforme NpPPOX2, S. cerevisiae Ygr017w; or Renalases such as Homo sapiens Renalase, modified forms of H. sapiens Renalase such as m5hRen1 or m6hRen1, etc. An NADPH detoxifying enzyme may comprise a tag used for the detection or immobilization to a solid support, including, without limitation, polyhistidine or a variant thereof, FLAG, glutathione S-transferase (GST), green florescent protein (GFP) or a variant thereof, biotin, hemagglutinin (HA), maltose binding protein (MBP), Myc, small ubiquitin like modifier (SUMO), ubiquitin, etc. In some embodiments, flavin mononucleotide (FMN) is added to the immobilized NADPH detoxification enzyme after it has been immobilized (i.e. attached). In some embodiments, flavin adenine dinucleotide (FAD) is added to the immobilized NADPH detoxification enzyme after it has been immobilized (i.e. attached). If a resin is used as a substrate, the resin may be any size deemed necessary. For instance, the resin may be in the diameter range of 30-200 μm to ensure acceptable flow properties.

In an aspect, the disclosure provides a NADPH detoxification device. The detoxification device comprises an input line, a chamber comprising one or a cocktail of NADPH detoxifying enzymes, and an output line, wherein the input line and output lines connect the chamber with other process devices. A cell-free reaction fluid, e.g. bioreactor fluid, comprising one or more of 1,2-NADPH, 1,4-NADPH and 1,6-NADPH is passed through the detoxification device. An oxygen introducing element can be attached to either the input line or the chamber. In some embodiments, the NADPH detoxifying enzymes are in solution within the chamber. In some embodiments, the NADPH detoxifying enzymes are immobilized on a substrate within the chamber. In some embodiments, the chamber further comprises a catalase enzyme, optionally immobilized on a substrate, that converts H₂O₂ side product produced from the detoxification reaction into H₂O and O₂. In some embodiments, a degassing element is attached to the output line to remove oxygen not used in the detoxification reaction. The detoxification device comprises NADPH detoxifying enzymes in amount effective to convert 1,2-NADPH and 1,-6-NADPH to NADP⁺, at a sufficient rate to maintain the desired productivity of a cell-free reaction. For example, the single pass conversion of 1,2-NADPH or 1,-6-NADPH to NADP⁺ may be in the range of from about 1 to about 100 μmol, from about 10 to about 100 μmol, from about 50 to 80 μmol, etc. Such conversion will be provided as determined to limit process metabolic rate reductions to less than 5%, less than 10%, or less than 20%.

In an embodiments, compositions and methods are provided for enzymatic, cell-free synthetic systems that produce reducing equivalents NADH and/or NADPH from H₂, which may find use in a bioreactor synthetic system. Cell-free metabolism and a simple secondary enzymatic pathway are used to harvest reducing equivalents from H₂ and to deliver the reducing equivalents to biosynthetic pathways, in the form of NADH or NADPH. The process may be conducted in substantially anaerobic conditions.

In some embodiments a bioreactor system is provided, which provides for delivery of H₂ to a main bioreactor chamber, e.g. for the production of commodity chemicals and the like. In one embodiment, a stream of bioreactor aqueous fluid, or a filtrate obtained from aqueous reactor fluid, is continuously removed from the bioreactor. That fluid is raised to a higher pressure, e.g. with a positive displacement pump, etc. to create a pressurized stream. Gaseous H₂ is injected into the pressurized stream. The injection is made through an injection element, e.g. nozzles with small orifices, etc., such that while small H₂ bubbles may be initially formed, there is sufficient residence time in the pressurized stream for the gaseous H₂ to be fully dissolved within the pressurized stream, thereby forming a supersaturated aqueous H₂ solution. A backpressure regulatory device may be used to maintain this stream at a constant total pressure. The supersaturated aqueous H₂ solution is reintroduced into the bioreactor to be distributed throughout the reactor volume, for example through one or more injection nozzles. In the bioreactor, the H₂ can reform gaseous bubbles, which results in the formation of bubbles of pure H₂ that provide a high chemical activity driving force for transfer from the gas phase into the aqueous phase, providing excellent mass transfer of hydrogen into the aqueous reactor fluid. In an alternative embodiment, gaseous H₂ is directly injected at high pressure into the bioreactor through one or many injection element(s).

The gaseous H₂ can be generated by any means, for example, through the use of an electrolyzer. When an electrolyzer is used, electricity is used to generate H₂ and O₂ from H₂O, where the O₂ is expelled from the anodic chamber and the H₂ is expelled from the cathodic chamber.

Enzymatic production of reducing equivalents, as disclosed herein, utilizes lysates or isolated enzymes derived from microbial cells, where the microbial cells express high levels of (i) active [FeFe] hydrogenase; (ii) ferredoxin; and (iii) ferredoxin-NADP-reductase (FNR) or ferredoxin-NAD⁺-reductase (FN⁺R). The microbial cells may be genetically modified to express or over-express one or more of these enzymes. The enzymes may be genetically modified from wild-type to alter O₂ tolerance, activity, specificity, etc. These enzymes may be referenced herein collectively as “NAD reducing pathway enzymes”. The NAD reducing pathway enzymes can be expressed in a single cell or in separate cells, but will generally be combined in the bioreactor.

The one or more microbial cells expressing NAD reducing pathway enzymes are lysed and the lysate, which is optionally a crude lysate, is combined with H₂ in a bioreactor during production phase, thereby producing one or both of NADH and NADPH. In that reaction, hydrogenase catalyzes the breakdown of H₂ to extract electrons, and passes the electrons to ferredoxin, to produce reduced ferredoxin. FNR or FN+R transfers electrons from reduced ferredoxin to NAD or NADP, thereby producing NADH or NADPH, and oxidized ferredoxin. The reaction mixture may be further supplemented with enzymatic cofactors or precursors thereof, as required. Preferred reaction conditions are substantially anaerobic in order to prevent inhibition or inactivation of O₂ sensitive hydrogenases and to avoid loss of reducing equivalents toward O₂ reduction. Desirably, cytochrome D oxidase is also present.

### BRIEF DESCRIPTION OF THE DRAWINGS

FIG. 1. A non-limiting example of an integrated system for the production of carbon negative commodity chemicals.

FIG. 2 illustrates a similar integrated system to FIG. 1 which additionally provides elements to supersaturate a stream of bioreactor fluid with H₂.

FIG. 3. A non-limiting example of a detoxification device.

FIG. 4. Succinate production from glucose and CO₂.

FIG. 5. NAD isomers and detoxification reactions.

FIG. 6. Addition of PNPO to a cell-free reaction increases cell-free reaction product production.

FIG. 7. Reaction scheme for the reduction of NAD⁺.

FIG. 8. Reaction scheme for the reduction of NADP⁺.

FIG. 9. Reaction scheme for the synthesis of NAD.

FIG. 10. A. Reaction scheme for the synthesis of CoenzymeA; B. Diagram of TCA

(tricarboxylic acid) Cycle.

FIG. 11. Reagents used for minimal cell-free protein synthesis system.

FIG. 12. sfGFP cell-free production as influenced by substrates used for the in situ synthesis of either NAD (Ribose and Nicotinamide) or CoenzymeA (Pantothenic Acid) or both cofactors.

### DETAILED DESCRIPTION OF THE EMBODIMENTS

All structural and functional equivalents to the features and method acts of the various embodiments described throughout the disclosure that are known or later come to be known to those of ordinary skill in the art are intended to be encompassed by the features described and claimed herein. Moreover, nothing disclosed herein is intended to be dedicated to the public regardless of whether such disclosure is explicitly recited in the claims. No claim element is to be construed under the provisions of 35 USC 112, sixth paragraph, unless the element is expressly recited using the phrase “means for” or “step for”.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Nicotinamide adenine dinucleotide (NAD). NAD is a coenzyme central to metabolism. Found in all living cells, NAD is called a dinucleotide because it consists of two nucleotides joined through their phosphate groups. One nucleotide contains an adenine nucleobase and the other nicotinamide. NAD exists in two forms: an oxidized and reduced form, abbreviated as NAD⁺ and NADH, respectively. In metabolism, nicotinamide adenine dinucleotide is involved in redox reactions, carrying electrons from one reaction to another. The cofactor is, therefore, found in two forms in cells: NAD⁺ is an oxidizing agent, it accepts electrons from other molecules and becomes reduced. This reaction, also with H⁺, forms NADH, which can then be used as a reducing agent to donate electrons. These electron transfer reactions are the main function of NAD.

NAD phosphate (NADP) is a cofactor used in anabolic reactions, such as the Calvin cycle and lipid and nucleic acid syntheses, which require NADPH as a reducing agent. NADPH is the reduced form of NADP⁺. NADP⁺ differs from NAD⁺ by the presence of an additional phosphate group on the 2′ position of the ribose ring that carries the adenine moiety. This extra phosphate is added by NAD⁺ kinase and removed by NADP⁺ phosphatase. NADP is used to refer to both NAD and NADP.

NAD and NADP are vital for cellular metabolism, but due to their intrinsically high chemical reactivity, they are particularly prone to be converted to aberrant and potentially toxic forms. Thus, NAD and NADP may undergo different types of aberrant reactions. One form of aberrant nicotinamide nucleotides are species where the reduced atom on the nicotinamide ring is C6 or C2 (1,6-NADPH and 1,2-NADPH) rather than C4 as in classical NADPH (1,4-NADPH). These forms arise spontaneously during long-term storage of NADPH solutions, resulting from the reduction of C6 or C2 of contaminating NADP+ by NADPH. 1,2-NADPH is highly unstable, whereas 1,6-NADPH is much more stable. Furthermore, 1,6-NADPH is a strong inhibitor of several dehydrogenases, including lactate dehydrogenase and malate dehydrogenase.

“Concomitant synthesis” means synthesis of one or more components, such as engineered proteins, commodity chemicals, cofactors, etc. at such time that the combination will have a beneficial effect. Such concomitant administration may involve concurrent (i.e. at the same time), prior, or subsequent synthesis of components.

The use of the term “in combination” does not restrict the order in which components are synthesized. A first component can be synthesizes prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) synthesis of a second component.

As used herein, “in vitro reaction” refers to a reaction performed in a controlled environment (e.g., an experimental environment or an environment outside a living organism).

As used herein, the terms “purified” and “isolated” when used in the context of an enzyme that is substantially free of contaminating materials from the material from which it was obtained, e.g. cellular materials, such as but not limited to cell debris, cell wall materials, membranes, organelles, the bulk of the nucleic acids, carbohydrates, proteins, and/or lipids present in cells. Thus, a polypeptide that is isolated includes preparations of a polypeptide having less than about 30%, 20%, 10%, 5%, 2%, or 1% (by dry weight) of cellular materials and/or contaminating materials. As used herein, the terms “purified” and “isolated” when used in the context of a enzyme that is chemically synthesized refers to a polypeptide which is substantially free of chemical precursors or other chemicals which are involved in the syntheses of the polypeptide.

As used herein, “cell-free” refers to a non-living system, e.g., in vitro or ex vivo systems containing cellular components. Sources for the components of cell-free systems include cell extracts and lysates, usually a crude cell lysate. As used herein a crude cell lysate comprises the lysate of a population of cells, which is substantially free of intact cells; and which is usually not subjected to enrichment techniques such as chromatography, dialysis, and the like. Such a cell lysate may or may not be clarified by such means as centrifugation or filtration. Cell-free systems are able to reconstitute cellular reactions, e.g., enzymatic and metabolic pathways. A cell lysate, usually a crude cell lysate, is obtained from cells expressly engineered to synthesize one or more proteins of interest, which results in the synthesis of a biomaterial of interest, for example, succinate.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

A “coding sequence” or a sequence which “encodes” a selected polypeptide, is a nucleic acid molecule which is transcribed to produce RNA (in the case of DNA) and translated (in the case of mRNA) into a polypeptide, for example, in a cell-free polypeptide synthesis reaction; or in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A transcription termination sequence may be located 3′ to the coding sequence. Other “control elements” may also be associated with a coding sequence.

“Operably linked” refers to an arrangement of elements wherein the components so described are configured so as to perform their usual function. Thus, a given promoter that is operably linked to a coding sequence (e.g., a reporter expression cassette) is capable of effecting the expression of the coding sequence when the proper enzymes are present. The promoter or other control elements need not be contiguous with the coding sequence, so long as they function to direct the expression thereof. For example, intervening un-translated yet transcribed sequences can be present between the promoter sequence and the coding sequence and the promoter sequence can still be considered “operably linked” to the coding sequence.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as integrating vectors

“Purified polynucleotide” refers to a polynucleotide of interest or fragment thereof which is essentially free, e.g., contains less than about 50%, preferably less than about 70%, and more preferably less than about 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

A “polypeptide” is used in it broadest sense to refer to a compound of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics.

Extract organism. As described above, the coding sequence for one or more enzymes utilized in the cell-free reaction are present or introduced into a source organism, which is used as a source of the proteins, e.g. as an extract or lysate. The coding sequences may be present on a replicable vector or inserted into the source organism genome using methods well-known to those of skill in the art. Such vector sequences are well known for a variety of bacteria. The expression vector may further comprise sequences providing for a selectable marker, induction of transcription, etc.

The coding sequences are operably linked to a promoter sequence active in the organism. Promoters are untranslated sequences located upstream (5′) to the start codon of a structural gene (generally within about 100 to 1000 bp) that control the transcription and translation of particular nucleic acid sequence. Promoters may be constitutive or inducible, where inducible promoters initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to protein-encoding DNA by removing the promoter from the source DNA, e.g. by PCR amplification of the sequence, etc. and inserting the isolated sequence into the vector. Both the native hydrogenase promoter sequence and many heterologous promoters may be used for expression, however, heterologous promoters are preferred, such as T7, as they generally permit greater transcription and higher yields. Promoters suitable for use with prokaryotic hosts include the β-lactamase and lactose promoter systems; alkaline phosphatase; a tryptophan (trp) promoter system; an arabinose promoter system; and hybrid promoters such as the tac promoter. However, other known bacterial and bacteriophage promoters are suitable. Their nucleotide sequences have been published, thereby enabling a skilled worker operably to ligate them to DNA encoding the NAD reducing pathway proteins.

Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

Host cells are transfected and preferably transformed with the above-described expression or cloning vectors and cultured in conventional nutrient media modified as appropriate for inducing promoters, selecting transformants, and preparing extracts as set forth in the Examples.

Sugar. As used herein, the term refers to a number of carbohydrates, such as monosaccharides, disaccharides, oligosaccharides, and polysaccharides, usually pentose or hexose sugars or polymers thereof. Monosaccharides that find use include, without limitation, fructose, arabinose, lyxose, ribose, xylose, ribulose, xylulose, deoxyribose, allose, altrose, glucose, mannose, gulose, idose, galactose, talose. Disaccharides may include sucrose, lactose, maltose, etc. Polysaccharides may include starches, glycogen, cellulose, pectin, peptidoglycan, lipopolysaccharides, capsules, exopolysaccharides, and the like. Sugars may be phosphorylated, e.g. glucose-6-phosphate, etc. Sugars may be included in the reaction mix at a concentration sufficient to provide energy for NAD reduction and synthesis of the desired biomaterial, e.g. from about 1 mM to about 1000 mM, and may be about 5 mM, 10 mM, 25 mM, 50 mM, 75 mM, 100 mM, 250 mM, 500 mM, 750 mM, 1000 mM. The sugars may also be fed into the reaction compartment to sustain metabolic and biosynthetic reactions.

Reaction mix: as used herein refers to a reaction mixture, present in an aqueous bioreactor fluid, capable of catalyzing the synthesis of reducing equivalents of NADH. The reaction mixture comprises extracts or lysates from bacterial cells, and the synthesis is performed under substantially anaerobic conditions. The volume percent of extract in the reaction mix will vary, where the extract is usually at least about 10% of the total volume; more usually at least about 20%; and in some instances may provide for additional benefit when provided at least about 50%; at least about 60%; or at least 75% of the total volume. In certain industrial applications the volume percent of extract may be around about 90% or higher. The reaction mixture may be further supplemented with one or more of niacin, nicotinamide, NAD, etc., usually at a concentration of from about 0.1 mM to 5 mM, e.g. at about 0.5 mM, about 1 mM, about 2 mM, about 3, etc. and may be from about 2 mM to about 5 mM, as a precursor or source of NAD; a nuclease, particularly a ribonuclease, which may be used at a conventional dose for example from about 0.5 μg/ml to about 50 μg/ml, to break down nucleic acids and generate adenine.

Before describing the present invention in detail, it is to be understood that this invention is not limited to particular formulations or method parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

### Bioreactors

Compositions and methods are provided for cell-free synthesis of reducing equivalents of NADH from H₂, using a bioreactor that provides for delivery of H₂ to a main bioreactor chamber. Within the main chamber, an enzymatic pathway composed of the following proteins: (i) active [FeFe] hydrogenase; (ii) ferredoxin; and (iii) ferredoxin-NADP⁺-reductase (FNR) or ferredoxin-NAD⁺-reductase (FN⁺R) is present. In the bioreactor, the enzyme pathway functions together with a biosynthetic pathway to transfer electrons from the H₂ to the oxidized NAD⁺ or NADP⁺ using ferredoxin as the electron carrier. The bioreactor system can be utilized in the synthesis of biomaterials with synthetic processes requiring NADH or NADPH. In some embodiments the biomaterials are commodity biomaterials.

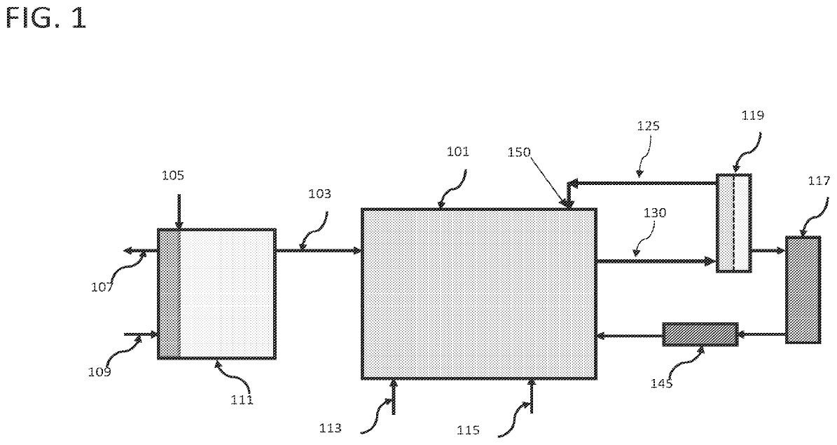

FIG. 1 illustrates one example of an overall integrated system, e.g. for the cell-free synthesis of reducing equivalents of NADH from H₂. In this embodiment, aqueous bioreactor fluid in the main chamber (101) comprises NAD reducing pathway enzymes comprising (i) [FeFe] hydrogenase; (ii) ferredoxin; and (iii) ferredoxin-NADP-reductase (FNR) or ferredoxin-NAD+-reductase (FN+R). Inputs into the main chamber include glucose (113), CO₂ (115), and highly pressurized H₂ (103). The aqueous bioreactor fluid output recycling stream (130) can circulate through an ultrafiltration device (119) that is able to separate desired biomaterials produced in the bioreactor from enzymes and other proteins, and this or a derivative stream (such as a filtrate) may be pumped through a capture device (117) to isolate and purify the desired biomaterials from the recycling stream. The bioreactor fluid input recycling stream (125) is returned to the main chamber through an inlet (150). The inlet (150) may comprise one or more nozzles or similar elements. The cell free recycling stream may also be pumped to a NADH detoxification device (145) in order to convert undesirable NAD byproducts such as 1,2-NADH and 1,6-NADH back to NAD⁺ before being returned to the cell free reactor (101), which embodiment aqueous bioreactor fluid in the main chamber (101) comprises NAD reducing pathway enzymes comprising (i) [FeFe] hydrogenase; (ii) ferredoxin; and (iii) ferredoxin-NADP-reductase (FNR) or ferredoxin-NAD+-reductase (FN+R).

The H₂ necessary for the synthesis of reducing equivalents of NAD/P(+) and the production of the desired biomaterials can be injected into the main chamber (101) using any means that provides a suitably high partial pressure of H₂ in the main chamber. This pressure will be maintained such that the rate of H₂ transfer into the liquid phase is at least 1 mmole/L-min, 2 mmoles/L-min or up to 5 mmoles/L-min or higher. In an embodiment, the H₂ stream (103) is injected into the cell free reactor from an electrolyzer (111). Electricity (105) and H₂O (109) are delivered to the electrolyzer which then utilizes said electricity to break down H₂O to produce H₂ and O₂. The O₂ is then released (107) from the electrolyzer while the H₂ is injected (103) at high pressure into the main chamber (101) through an injection element.

A degassing element may be included, where it is any element capable of removing most (i.e. >90%) of all of the oxygen within the cell-free reaction mixture (or cell-free recycling stream). For instance, the degassing element may be a pump, a vacuum, a membrane, etc.

As used herein, “electrolyzer” refers to a device or a system that intakes electricity and H₂O and uses said electricity to break the H₂O down into hydrogen and oxygen in a process known as electrolysis. Oxygen emerges from the anodic chamber and hydrogen from the cathodic chamber. Both gases can be produced at moderate to high pressures, The oxygen and hydrogen produced may then either be stored in pressure vessels for later use or used continuously. Examples of suitable electrolyzers that find use in the present disclosure, include without limitation, industrial scale units produced by companies such as Nel and smaller hydrogen generator units produced by companies such as VICI DBS. For example, the PG Plus 100 Hydrogen Generator from VICI DBS can produce pure hydrogen at 100 ml/min at pressures up to 160 psig. The industrial units typically allow the production and capture of both hydrogen and oxygen.

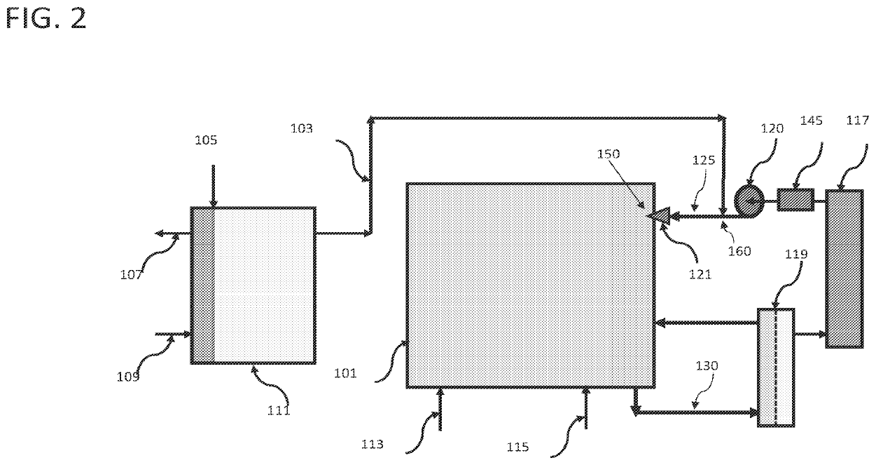

FIG. 2 illustrates a similar integrated system to FIG. 1, in which H₂ is delivered as a supersaturated aqueous solution, which is produced by injecting H₂ at high pressure into the bioreactor fluid input recycle stream. In this system, the H₂ stream (103) from an electrolyzer (111) is injected into the bioreactor fluid input recycling stream (125). The input recycling stream (125) is pressurized, for example with a positive displacement pump (120), and the H₂ stream is injected into the pressurized bioreactor fluid input recycling stream. The H₂ stream injection element (160) for this purpose may be, for example, a nozzle. The resulting bioreactor fluid input recycling stream is a two phase fluid, which will then reside in the return pipe (125) or in a holding loop until the gaseous H₂ has transferred into the liquid phase. A back pressure control device (121) maintains the higher pressure in the return pipe or holding loop. The back pressure control device (121) is installed between the positive displacement pump (125) and the inlet (150) into the bioreactor main chamber. The back pressure regulator maintains the same pressure in the holding loop independent of flow rate, providing a benefit over elements where the pressure would depend on the rate of ejection into the bioreactor fluid and therefore on the fluid recirculation rate which could vary.

The resulting input stream (125) is a single phase liquid that is supersaturated with H₂. When this fluid is injected into the main chamber, multiple small bubbles of pure H₂ gas are formed to provide high mass transfer (with the same rates or higher as described for FIG. 1) into the low pressure fluid in the bioreactor. The bubbles may be expected to be smaller and surface tension effects could maintain a higher pressure in the smaller bubbles to increase the transfer driving force. Both the large quantity and very small size of the H₂ bubbles contribute to supplying sufficient mass transfer of H₂ into the cell free reactor to support the required production rate of NADH. Such arrangements are highly advantageous in avoiding the expense of supplying reaction vessels with high pressure ratings.

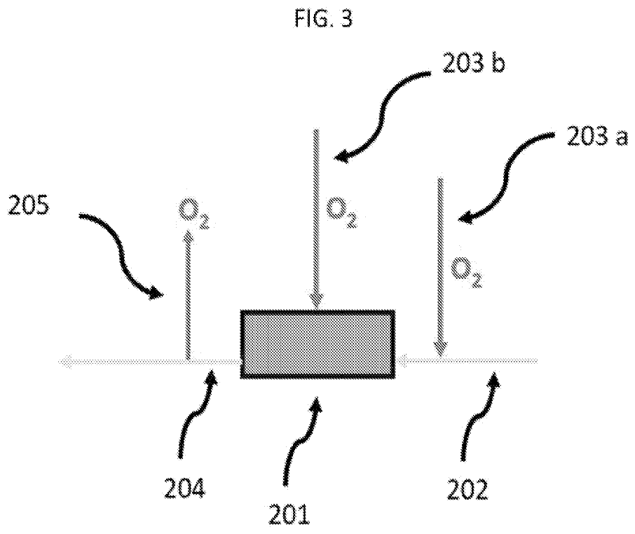

FIG. 3 illustrates an exemplary NAD detoxification device. In this embodiment, a cell-free reaction mixture (or a cell-free recycling stream) comprising a mixture of NADP tautomers is introduced to an input line (202) which is connected to a chamber comprising one or a cocktail of NADPH detoxifying enzymes (201). In some embodiments, an oxygen (O₂) introducing element (203a) is attached to the input line. In some embodiments, an oxygen (O₂) introducing element (203b) is attached to the chamber. 1,2-NADPH and 1,6-NADPH present in the recycling stream are oxidized by the NADPH detoxifying enzymes, to generate NADP⁺. In some embodiments, the chamber further comprises catalase enzymes that convert H₂O₂ side products produced from the detoxification reaction into H₂O and O₂. In some embodiments, the chamber further comprises flavin mononucleotide (FMN) or flavin adenine dinucleotide (FAD). The recycling stream, comprising 1,4-NADPH and/or NADP⁺ then exits the chamber through an output line (204). In some embodiments, a degassing element (205) attached to the output line (204) removes O₂ from the recycling stream, following the exit from the chamber comprising NADPH detoxifying enzymes.

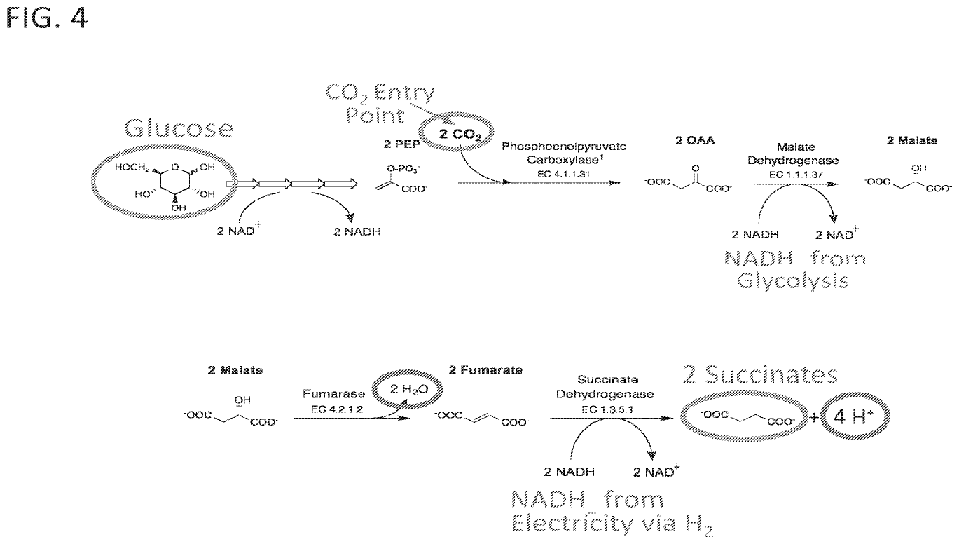

FIG. 4 illustrates an exemplary overall metabolic scheme for production of a desired biomaterial, using succinate as a non-limiting example. One molecule of glucose is combined with two molecules of CO₂ to produce two molecules of the product, succinate. Direct CO₂ incorporation into a new biomolecule is particularly desirable, and this requires additional NADH supplied by using H₂ as the electron source.

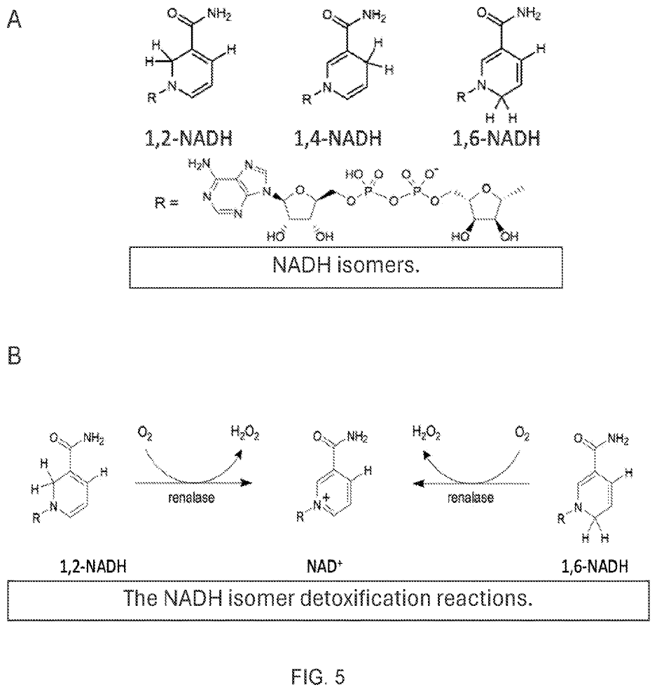

FIG. 5A-5B illustrates the overall NAD detoxification scheme and tautomers. Renalases, PNPOs or PNPO-RPs oxidize either 1,2-NADPH or 1,6-NADPH using O₂ producing NADP⁺ and H₂O₂.

### Enzymes

In the various embodiments of the disclosure, enzymes are used, e.g. for oxidation of hydrogen, detoxification of NAD tautomers, and synthesis of cofactors. A description of enzymes of interest is provided below.

Enzymes may be isolated and purified in accordance with conventional methods of recombinant synthesis or cell free protein synthesis. Separation procedures of interest include affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural biospecific binding of one molecular species to separate and purify a second species from a mixture. Antibodies are commonly used in affinity chromatography. Preferably a microsphere or matrix is used as the support for affinity chromatography. Such supports are known in the art and are commercially available, and include activated supports that can be combined to the linker molecules. For example, Affi-Gel supports, based on agarose or polyacrylamide are low pressure gels suitable for most laboratory-scale purifications with a peristaltic pump or gravity flow elution. Affi-Prep supports, based on a pressure-stable macroporous polymer, are suitable for preparative and process scale applications. Enzymes may also be separated by ion exchange chromatography, and/or concentrated, filtered, dialyzed, etc., using methods known in the art.

Various methods can be used to immobilize enzymes, e.g. renalase, PNPO, catalase, etc. to a substrate. For example, the enzyme can be joined through a linker, e.g. a polypeptide linker, or a non-peptidic linker, etc. Chemical groups that find use in linking include carbamate; amide (amine plus carboxylic acid); ester (alcohol plus carboxylic acid), thioether (haloalkane plus sulfhydryl; maleimide plus sulfhydryl), Schiff's base (amine plus aldehyde), urea (amine plus isocyanate), thiourea (amine plus isothiocyanate), sulfonamide (amine plus sulfonyl chloride), disulfide; hydrazone, lipids, and the like, as known in the art.

The linkage between an enzyme and substrate may comprise spacers, e.g. alkyl spacers, which may be linear or branched, usually linear, and may include one or more unsaturated bonds; usually having from one to about 300 carbon atoms; more usually from about one to 25 carbon atoms; and may be from about three to 12 carbon atoms. Spacers of this type may also comprise heteroatoms or functional groups, including amines, ethers, phosphodiesters, and the like.

The enzymes may be immobilized through a homo- or heterobifunctional linker having a group at one end capable of forming a stable linkage to the hydrophilic head group, and a group at the opposite end capable of forming a stable linkage to the targeting moiety. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-γ-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, NHS-PEG-MAL; succinimidyl 4-[N-maleimidomethyl]cyclohexane-1-carboxylate; 3-(2-pyridyldithio) propionic acid N-hydroxysuccinimide ester (SPDP); N,N′-(1,3-phenylene) bismaleimide; N,N′-ethylene-bis-(iodoacetamide); 4-(N-maleimidomethyl)-cyclohexane-1-carboxylic acid N-hydroxysuccinimide ester (SMCC); m-maleimidobenzoyl-N-hydroxysuccinimide ester (MBS), and succinimide 4-(p-maleimidophenyl) butyrate (SMPB), an extended chain analog of MBS. The succinimidyl group of these cross-linkers reacts with a primary amine, and the thiol-reactive maleimide forms a covalent bond with the thiol of a cysteine residue.

Other reagents useful for this purpose include: p,p′-difluoro-m,m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate or azophenyl-p-diisocyanate (which reacts principally with amino groups); disdiazobenzidine (which reacts primarily with tyrosine and histidine); O-benzotriazolyloxy tetramethyluronium hexafluorophosphate (HATU), dicyclohexyl carbodimide, bromo-tris (pyrrolidino) phosphonium bromide (PyBroP); N,N-dimethylamino pyridine (DMAP); 4-pyrrolidino pyridine; N-hydroxy benzotriazole; and the like. Homobifunctional cross-linking reagents include bismaleimidohexane (“BMH”).

Alternatively the enzyme of interest may comprise at least one unnatural amino acid at a pre-determined site, and may comprise or contain 1, 2, 3, 4, 5 or more unnatural amino acids. If present at two or more sites in the polypeptide, the unnatural amino acids can be the same or different. Examples of unnatural amino acids that can be used include: an unnatural analogue of a tyrosine amino acid; an unnatural analogue of a glutamine amino acid; an unnatural analogue of a phenylalanine amino acid; an unnatural analogue of a methionine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; a cyclic amino acid other than proline, etc.

Unnatural amino acids of interest include, without limitation, amino acids that provide a reactant group for CLICK chemistry reactions (see Click Chemistry: Diverse Chemical Function from a Few Good Reactions Hartmuth C. Kolb, M. G. Finn, K. Barry Sharpless Angewandte Chemie International Edition Volume 40, 2001, P. 2004, herein specifically incorporated by reference). For example, the amino acids azidohomoalanine, homopropargylglycine, p-acetyl-L-phenylalanine and p-azido-L-phenylalanine are of interest.

An unnatural amino acid is introduced by global replacement of methionine on the protein, e.g. methionine can be left out of a cell-free reaction mixture, and substituted by from 0.25-2.5 mM azidohomoalanine (AHA). Alternatively an unnatural amino acid is introduced by orthogonal components. Orthogonal components include a tRNA aminoacylated with an unnatural amino acid, where the orthogonal tRNA base pairs with a codon that is not normally associated with an amino acid, e.g. a stop codon; a 4 bp codon, etc. An in vitro reaction mixture to synthesize the enzyme of interest may further comprise a tRNA synthetase capable of aminoacylating (with an unnatural amino acid) the cognate orthogonal tRNA. Such components are known in the art, for example as described in U.S. Pat. No. 7,045,337, issued May 16, 2006. The orthogonal tRNA recognizes a selector codon, which may be nonsense codons, such as, stop codons, e.g., amber, ochre, and opal codons; four or more base codons; codons derived from natural or unnatural base pairs and the like. The orthogonal tRNA anticodon loop recognizes the selector codon on the mRNA and incorporates the unnatural amino acid at this site in the polypeptide.

A substrate may comprise a complementary active group for CLICK chemistry conjugation, for example a substrate may comprise on it's surface with one or more unnatural amino acids that allow for the conjugation to the unnatural amino acid present on the enzyme of interest. For example, a substrate may display alkynes allowing linkage of enzymes displaying azides. Methods of immobilizing on a substrate may take advantage of the reactive groups in non-natural amino acids to provide a linkage, e.g. a covalent linkage.

### Synthetic Enzymes

Ribokinase. Ribokinase belongs to the phosphofructokinase B family of sugar kinases. Ribokinases fall under the enzyme commission number of EC 2.7.1.15. Ribokinase in both prokaryotes and eukaryotes catabolizes ribose by conversion into ribose 5-phosphate via a phosphoryl transfer from ATP to the O-5′ position on ribose. The equivalent chemical reaction is as follows:

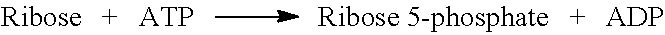

Phosphoribosyl pyrophosphate synthetase. Phosphoribosyl-pyrophosphate synthetase (PRS; also known as ribose-phosphate diphosphokinase) catalyzes the transfer of the terminal pyrophosphoryl group of ATP to ribose 5-phosphate to generate phosphoribosyl pyrophosphate (PRPP). PRSs fall under the enzyme commission number of EC 2.7.6.1. PRSs are characterized by the ability to catalyze the following chemical reaction:

Ribose 5-phosphate+ATP→phosphoribosyl pyrophosphate(PRPP)+AMP

Nicotinamide phosphoribosyltransferase (NAMPT). NAMPT catalyzes the condensation of nicotinamide with phosphoribosyl pyrophosphate to yield nicotinamide mononucleotide, an intermediate in the biosynthesis of NAD⁺. NAMPTs fall under the enzyme commission the number of EC 2.4.2.12. NAMPTs are characterized by the ability to catalyze the following chemical reaction:

Nicotinamide+PRPP→nicotinamide mononucleotide(NMN)+pyrophosphate(PPi)

Nicotinamide-nucleotide adenylyltransferase (NMNAT). NMATs fall under the enzyme commission the number of EC 2.7.7.1. NMATs catalyze the formation of NAD⁺ from nicotinamide mononucleotide (NMN) and ATP according to the following reaction:

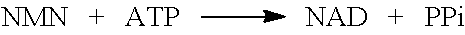

### Hydrogenases and Reductases

O₂ tolerance. As used herein, the term O₂ tolerance refers to the ability of a hydrogenase enzyme to retain activity in the presence of molecular oxygen. As is known in the art, even trace levels of O₂ can be sufficient to inactivate known hydrogenases. Enzymes that have been mutated to increase tolerance may have at least about 10% activity, at least about 20% activity, at least about 30% activity or more after a solution of the protein is exposed to 0.05 atm O₂ for one hour. The enzymes may be more resistant to O₂ when the enzyme is present in a microbial host cell (relative to a solution).

Hydrogenase. Hydrogenases catalyze the reversible oxidation/reduction of molecular hydrogen (H₂) and play a vital role in anaerobic metabolism. Metal containing hydrogenases are subdivided into three classes: Fe—Fe hydrogenases, Ni—Fe hydrogenases, and Fe hydrogenases. Hydrogen oxidation is coupled to the reduction of electron acceptors such as oxygen, nitrate, sulphate, carbon dioxide and fumarate and may also be coupled to electron carriers such as ferredoxins, whereas proton reduction (H₂ evolution) is typically coupled to molecules such as ferredoxin. A hydrogenase of interest for the present invention catalyzes the breakdown of H₂ to extract electrons, and passes the electrons to ferredoxin, to produce reduced ferredoxin.

In one embodiment, the term “hydrogenase” as used herein refers to an enzyme that meets one or more of the criteria provided herein. Using these criteria, one of skill in the art can determine the suitability of a candidate enzyme for use in the methods of the invention. Many enzymes will meet multiple criteria, including two, three, four or more of the criteria, and some enzymes will meet all of the criteria. The terms “hydrogenase” can refer to a full length enzyme or fragment thereof with the capability of catalyzing hydrogen oxidation/reduction.

Hydrogenases of the invention include enzymes having at least about 20% sequence identity at the amino acid level, more usually at least about 40% sequence identity, and preferably at least about 70% sequence identity to one of the following hydrogenases: Chlamydomonas reinhardtii iron-iron-hydrogenase (Genbank accession AY055756); Clostridium pasteurianum hydrogenase (Cpl, Genbank accession AAA23248.1); Megasphaera elsdenii hydrogenase (Genbank accession AF120457); Desulfovibrio vulgaris hydrogenase (Genbank accession CAA26266.1). For example, see Forestier et al. (2003) Eur. J. Biochem. 270 (13), 2750-2758; Meyer et al. (1991) Biochemistry 30:9697-9704; Voordouw et al. (1985) Eur. J. Biochem. 148:515-520; Atta et al. (2000) Biochim Biophys Acta. 1476 (2): 368-71; Fauque et al. (1988) FEMS Microbiol. Rev. 4, 299-344; Cammack et al. (1994) Methods Enzymol. 243, 43-68; and de Lacey et al. (1997) J. Am. Chem. Soc. 119, 7181-7189, each herein incorporated by reference. In some embodiments, the hydrogenase is an oxygen tolerant hydrogenase. Examples of oxygen tolerant hydrogenases are disclosed in U.S. Pat. No. 10,221,401, herein incorporated by reference.

Homology-based identification (for example, by a PILEUP sequence analysis) of enzymes can be routinely performed by those of skill in the art upon contemplation of this disclosure to identify those suitable for use in the methods of the present invention. Such enzymes are usually produced in microorganisms, particularly bacteria. Hydrogenases of the invention also include an enzyme belonging to the enzyme classifications EC 1.12.7.2 and EC 1.12.2.1.

The nucleic acid sequences encoding the above hydrogenases may be accessed from public databases as previously cited. Identification of additional hydrogenases is accomplished by conventional screening methods of DNA libraries or biological samples for DNA sequences having a high degree of similarity to known hydrogenase sequences.

Iron-iron hydrogenase. The hydrogenases containing no other metal than Fe and containing an active site H-cluster consisting of a 4Fe-4S subcluster joined by a cysteine residue to a 2Fe-2S cluster are called Fe—Fe hydrogenases ([Fe—Fe] Hases). Two families of Fe—Fe Hases have been described. Cytoplasmic, soluble, monomeric Fe—Fe Hases are found in strict anaerobes such as Clostridium pasteurianum and Megasphaera elsdenii. They are extremely sensitive to inactivation by O₂ and catalyze both H₂ evolution and uptake. Periplasmic, heterodimeric (Fe—Fe) Hases from Desulfovibrio spp., can be purified aerobically but catalyze mainly H₂ oxidation.

3-D structures of H₂ evolving (Fe—Fe) Hase I from Clostridium pasteurianum (Cpl) and Desulfovibrio desulfuricans uptake hydrogenase (DdH) are known. The overall structure of Cpl resembles a mushroom consisting of four domains: the large active site domain forms “cap” and three smaller domains form “stem”. The “stem” domains bind four iron-sulphur clusters and are termed FS4A-FS4B, FS4C and FS2. The N-terminal FS2 domain binds a [Fe₂S₂] cluster and shares the overall fold with plant-type ferredoxins. The FS4A-FS4B domain is adjacent to the active site domain; it contains two [Fe₄S₄] clusters and has the overall fold similar to that of bacterial type ferredoxins. The FS4C domain is placed between the FS2 and FS4A-FS4B domains and consists of two α-helices linked by a loop that binds a single [Fe₄S₄] cluster via one His and three Cys residues. The large subunit of DdH lacks FS4C and FS2 clusters and corresponding domains. The small subunit of DdH has an unusual fold consisting of alternating random coil and four α-helices that form a “belt” around the large subunit.

The active site domain of the (Fe—Fe) Hases contains an unusual Fe—S center termed the H-cluster. H-cluster consists of the [Fe₄S₄] subcluster bridged via the Cys thiolate to the [Fe₂] (binuclear iron-iron) subcluster. The two iron-iron atoms are designated Fe1 and Fe2 (proximal and distal with respect to the [Fe₄S₄] subcluster) and are ~2.6 Å apart. With the exception of the bridging Cys, the diiron-iron subcluster is coordinated by non-protein ligands. In Cpl, both iron-iron atoms are octahedrally coordinated to five CO/CN ligands, three S ligands and one water molecule. Fe1 and Fe2 are bridged by two S atoms and one CO or CN ligand. The two bridging sulphurs themselves can be bridged by a C—N—C connection. In DdH, Fe1 and Fe2 are bridged by a small molecule that has been modelled as 1,3-propanedithiol (PDT). Fe1 is octahedrally coordinated while Fe2 has square pyramidal coordination geometry.

Ferredoxin NAD/P(+) reductases (FN⁺R/FNR). Ferredoxin NAD/P(+) reductases catalyze the reversible oxidation/reduction of Nicotinamide adenine dinucleotide (NADH) or Nicotinamide adenine dinucleotide phosphate (NADPH). These enzymes can use two types of ferredoxins, the first carrying one electron and the second carrying two. The following reactions indicate catalysis by FN⁺R and FNR, respectively, using the latter type of ferredoxin:

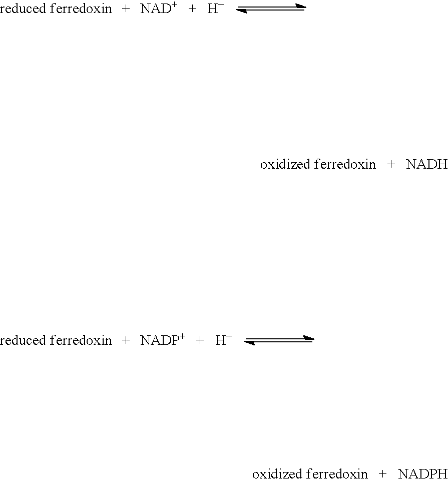

These enzymes belong to the family of oxidoreductases that use iron-sulfur proteins as electron donors and NAD+ or NADP⁺ as electron acceptors. These enzymes participate in photosynthesis and they provide a major source of NADPH or NADH in photosynthetic organisms. During photosynthesis, electrons are removed from water and transferred to the single electron carrier ferredoxin. Ferredoxin: NAD/P(+) reductase then transfers an electron from each of two ferredoxin molecules to a single molecule of the two electron carrier NADPH or NADH. FNR utilizes Flavin adenine dinucleotide (FAD), which can exist in an oxidized state, single electron reduced semiquinone state, and fully reduced state to mediate this electron transfer. Many similar reaction sequences are known and may proceed for purposes of either supplying or removing reducing equivalents. The methods of the invention may be applied to any of the Ferredoxin NAD/P(+) reductases that accept electrons from a ferredoxin. Table 1 provides representative examples.

In one embodiment, the term “Ferredoxin NAD/P(+) reductase” as used herein refers to an enzyme that meets one or more of the criteria provided herein. Using these criteria, one of skill in the art can determine the suitability of a candidate enzyme for use in the methods of the invention. Many enzymes will meet multiple criteria, including two, three, four or more of the criteria, and some enzymes will meet all of the criteria. The terms “Ferredoxin NAD/P(+) reductases” can refer to a full length enzyme or fragment thereof with the capability of catalyzing hydrogen oxidation/reduction.

TABLE 1

| Name | Gene | Species | UniprotKB |
|---|---|---|---|
| Anthranilate 1,2-dioxygenase | andAa | Burkholderia cepacia | Q84BZ0 |
| system ferredoxin--NAD(+) | | (Pseudomonas cepacia) | |
| reductase component | | | |
| Benzoate 1,2-dioxygenase | benC | Acinetobacter baylyi | P07771 |
| electron transfer component | | (strain ATCC 33305 / | |
| | | BD413 / ADP1) | |
| Biphenyl dioxygenase system | bphG | Paraburkholderia | P37337 |
| ferredoxin--NAD(+) reductase | | xenovorans (strain | |
| component | | LB400) | |
| p-cumate 2,3-dioxygenase | cmtAa | Pseudomonas putida | Q51973 |
| system, ferredoxin--NAD(+) | | (Arthrobacter | |
| reductase component | | siderocapsulatus) | |
| Dicamba O-demethylase 2, | ddmA2 | Stenotrophomonas | Q5S3I1 |
| ferredoxin reductase | | maltophilia | |
| component | | (Pseudomonas | |
| | | maltophilia) | |
| | | (Xanthomonas | |
| | | maltophilia) | |
| Ferredoxin--NAD(+) reductase | N/A | Streptomyces griseus | P24134 |
| 3-phenylpropionate/cinnamic | hcaD | Escherichia coli O157:H7 | Q8XA71 |
| acid dioxygenase ferredoxin-- | | | |
| NAD(+) reductase component | | | |
| 3-phenylpropionate/cinnamic | hcaD | Escherichia coli (strain | C4ZXB7 |
| acid dioxygenase ferredoxin-- | | K12 / MC4100 / BW2952) | |
| NAD(+) reductase component | | | |
| 3-phenylpropionate/cinnamic | hcaD | Escherichia coli (strain | B1IVT5 |
| acid dioxygenase ferredoxin-- | | ATCC 8739 / DSM 1576 / | |
| NAD(+) reductase component | | NBRC 3972 / NCIMB | |
| | | 8545 / WDCM 00012 / | |
| | | Crooks) | |
| 3-phenylpropionate/cinnamic | hcaD | Escherichia coli (strain | B6I5B5 |
| acid dioxygenase ferredoxin-- | | SE11) | |
| NAD(+) reductase component | | | |
| 3-phenylpropionate/cinnamic | hcaD | Shigella flexneri serotype | Q0T1X7 |
| acid dioxygenase ferredoxin-- | | 5b (strain 8401) | |
| NAD(+) reductase component | | | |
| Toluene 1,2-dioxygenase | todA | Pseudomonas putida | A5W4E9 |
| system ferredoxin--NAD(+) | | (strain ATCC 700007 / | |
| reductase component | | DSM 6899 / BCRC 17059 / | |
| | | F1) | |
| Xylene monooxygenase | xylA | Pseudomonas putida | P21394 |
| electron transfer component | | (Arthrobacter | |
| | | siderocapsulatus) | |
| Anthranilate 1,2-dioxygenase | antC | Acinetobacter baylyi | O85675 |
| electron transfer component | | (strain ATCC 33305 / | |
| | | BD413 / ADP1) | |
| Benzene 1,2-dioxygenase | bnzD | Pseudomonas putida | P08087 |
| system ferredoxin--NAD(+) | | (Arthrobacter | |
| reductase component | | siderocapsulatus) | |
| Ferredoxin--NAD(P)(+) | carAd | Pseudomonas | Q8GI14 |
| reductase CarAd | | resinovorans | |
| Chloroacetanilide N- | cndC1 | Sphingomonas wittichii | X5CY81 |
| alkylformylase, ferredoxin | | (strain DC-6 / KACC | |
| reductase component | | 16600) | |
| 2,4-dinitrotoluene dioxygenase | dntAa | Burkholderia sp. (strain | Q45692 |
| system ferredoxin--NAD(+), | | RASC) | |
| reductase component | | | |
| 3-phenylpropionate/cinnamic | hcaD | Escherichia coli | A7ZPY5 |
| acid dioxygenase ferredoxin-- | | O139:H28 (strain | |
| NAD(+) reductase component | | E24377A / ETEC) | |
| 3-phenylpropionate/cinnamic | hcaD | Escherichia coli O157:H7 | B5Z115 |
| acid dioxygenase ferredoxin-- | | (strain EC4115 / EHEC) | |
| NAD(+) reductase component | | | |
| 3-phenylpropionate/cinnamic | hcaD | Escherichia coli (strain | B1XB17 |
| acid dioxygenase ferredoxin-- | | K12 / DH10B) | |
| NAD(+) reductase component | | | |
| 3-phenylpropionate/cinnamic | hcaD | Escherichia coli (strain | P77650 |
| acid dioxygenase ferredoxin-- | | K12) | |
| NAD(+) reductase component | | | |
| 3-phenylpropionate/cinnamic | hcaD | Escherichia coli (strain | B1LNJ8 |
| acid dioxygenase ferredoxin-- | | SMS-3-5 / SECEC) | |
| NAD(+) reductase component | | | |
| 3-phenylpropionate/cinnamic | hcaD | Shigella sonnei (strain | Q3YZ11 |
| acid dioxygenase ferredoxin-- | | Ss046) | |
| NAD(+) reductase component | | | |
| Toluene 1,2-dioxygenase | todA | Pseudomonas putida | P0C621 |
| system ferredoxin--NAD(+) | | (Arthrobacter | |
| reductase component | | siderocapsulatus) | |
| Toluate 1,2-dioxygenase | xylZ | Pseudomonas putida | P23101 |
| electron transfer component | | (Arthrobacter | |
| | | siderocapsulatus) | |
| Benzene 1,2-dioxygenase | bedA | Pseudomonas putida | Q07946 |
| system ferredoxin--NAD(+) | | (Arthrobacter | |
| reductase subunit | | siderocapsulatus) | |
| Biphenyl 2,3-dioxygenase, | bphA4 | Rhodococcus jostii (strain | Q0S032 |
| ferredoxin reductase | | RHA1) | |
| component | | | |
| 2-halobenzoate 1,2- | cbdC | Burkholderia cepacia | Q51603 |
| dioxygenase electron transfer | | (Pseudomonas cepacia) | |
| component | | | |
| Dicamba O-demethylase 1, | ddmA1 | Stenotrophomonas | Q5S3I2 |
| ferredoxin reductase | | maltophilia | |
| component | | (Pseudomonas | |
| | | maltophilia) | |
| | | (Xanthomonas | |
| | | maltophilia) | |
| Ferredoxin--NAD(P)(+) | Fdr | Sphingomonas sp. | D5IGG6 |
| reductase fdr | | | |
| 3-phenylpropionate/cinnamic | hcaD | Escherichia coli (strain | B7LDD4 |
| acid dioxygenase ferredoxin-- | | 55989 / EAEC) | |
| NAD(+) reductase component | | | |
| 3-phenylpropionate/cinnamic | hcaD | Escherichia coli O7:K1 | B7NRJ1 |
| acid dioxygenase ferredoxin-- | | (strain IAI39 / ExPEC) | |
| NAD(+) reductase component | | | |
| 3-phenylpropionate/cinnamic | hcaD | Escherichia coli O9:H4 | A8A348 |
| acid dioxygenase ferredoxin-- | | (strain HS) | |
| NAD(+) reductase component | | | |
| 3-phenylpropionate/cinnamic | hcaD | Escherichia coli | B7N6C9 |
| acid dioxygenase ferredoxin-- | | O17:K52:H18 (strain | |
| NAD(+) reductase component | | UMN026 / ExPEC) | |
| 3-phenylpropionate/cinnamic | hcaD | Photorhabdus laumondii | Q7N4V5 |
| acid dioxygenase ferredoxin-- | | subsp. laumondii (strain | |
| NAD(+) reductase component | | DSM 15139 / CIP 105565 / | |
| | | TT01) | |
| Toluene-4-monooxygenase | tmoF | Pseudomonas mendocina | Q03304 |
| system, ferredoxin--NAD(+) | | | |
| reductase component | | | |
| Alkene monooxygenase | xamoF | Xanthobacter | A7IPX7 |
| system, ferredoxin--NAD(+) | | autotrophicus (strain | |
| reductase component | | ATCC BAA-1158 / Py2) | |

Ferredoxin (Fd) are ubiquitous, small proteins containing one or two iron-sulfur clusters. These prosthetic groups contain iron and sulfur atoms, organized in three different types of centers: 2Fe-2S, 4Fe-4 S, and 3Fe-4S. Despite the type and number of clusters bound, Fds generally function in oxidoreduction reactions. Many Fds are involved as electron carriers of low redox potential in electron transport chains of fundamental metabolic processes such as photosynthesis, nitrogen fixation, and assimilation of hydrogen, nitrogen, and sulfur. Bacteria generally harbor many Fds, which differ in amino acid sequence, FeS cluster type, redox potential, and function. Generally a ferrodoxin will be selected that is compatible with the FNR enzyme.

### Detoxifying Enzymes

NADPH detoxifying enzymes. NADPH detoxifying enzymes selectively oxidize 1,2-NADPH and/or 1,6-NADPH but not 1,4-NADPH using O₂, thereby producing NADP⁺ and H₂O₂. NADPH detoxifying enzymes general either fall under the categories of pyridoxamine-phosphate oxidases (PNPOs), PNPO-related proteins (PNPO-RPs), or Renalases. The methods can utilize one or a cocktail of different enzymes, e.g. 1, 2, 3, 4, or more different enzymes.

In one embodiment, the term “NADPH detoxifying enzymes” as used herein refers to an enzyme that meets one or more of the criteria provided herein. Using these criteria, one of skill in the art can determine the suitability of a candidate enzyme for use in the methods of the invention. Many enzymes will meet multiple criteria, including oxidizing 1,2-NADPH, oxidizing 1,6-NADPH, oxidizing both 1,2-NADPH and 1,6-NADPH or not oxidizing 1,4-NADPH, falling under the categories of the above mentioned enzymes, and some enzymes will meet all of the criteria. The terms “NADPH detoxifying enzymes” can refer to a full length enzyme or fragment thereof with the capability of catalyzing hydrogen oxidation/reduction.

NADPH detoxifying enzymes of the invention include enzymes having at least about 20% sequence identity at the amino acid level, more usually at least about 40% sequence identity, and at least about 70% sequence, at least about 90% identity to one of the following NADPH detoxifying enzymes: M. musculus (NM_134021.2), S. cerevisiae PNPO (Pdx3; NM_001178383.1); E. coli PNPO (PdxH; NC_000913.3); A. thaliana PNPO (PPOX1; NM_124376.4); A. thaliana PNPO-RP (PPOX2; NM_130223.3); S. cerevisiae PNPO-RP (Ygr017w; NM_001181146.1); N. punctiforme PNPO-RP (WP_012411992.1), H. sapiens renalase (RNLS; NM_001031709.3); H. sapiens modified renalase such as m5hRen1 or m6hRen1. For example, see Marbaix et al. Biochem J. 2019 Oct. 30; 476(20):3033-3052. and Morrison et al PLoS One. 2020 Nov. 12; 15(11):e0242109, each herein incorporated by reference.

The NADPH detoxifying enzyme may comprise a tag used for the detection or immobilization to a solid support. The tag may be any deemed beneficial. For instance, the tag may include, without limitation, polyhistidine or a variant thereof, FLAG, glutathione S-transferase (GST), green florescent protein (GFP) or a variant thereof, biotin, hemagglutinin (HA), maltose binding protein (MBP), Myc, small ubiquitin like modifier (SUMO), ubiquitin, etc.

Homology-based identification (for example, by a PILEUP sequence analysis) of enzymes can be routinely performed by those of skill in the art upon contemplation of this disclosure to identify those suitable for use in the methods of the present invention. Such enzymes are usually produced in microorganisms, particularly bacteria. NADPH detoxifying enzymes of the invention also include an enzyme belonging to the enzyme classifications EC: 1.4.3.5 and EC: 1.6.3.5.

Pyridoxamine-phosphate oxidase. In some embodiments an NADP detoxifying enzyme is a PNPO. PNPOs traditionally catalyse the direct transfer of a pair of electrons, in the form of hydride ion, from the C4′ of pyridoxine 5′-phosphate (PNP) or pyridoxamine 5′-phosphate (PMP) to a tightly bound molecule of FMN, forming FMNH₂. These electrons are then transferred in a second half-reaction to molecular oxygen, regenerating FMN and forming H₂O₂. PNPOs ability to oxidize 6-NADPH is highly conserved among PNPOs. More distantly related PNPOs designated PNPO-RPs display high catalytic efficiencies when oxidizing 1,6-NADPH and 1,2-NADPH but display no activity with 1,4-NADPH, PNP, or PMP.

Renalase. In some embodiments an NADP detoxifying enzyme is a renalase. Renalase (RNLS) is a small flavoprotein produced mainly by the kidney. RNLS shows both intracellular and extracellular activity. Intracellular RNLS acts as an enzyme that, in the presence of a flavin adenine dinucleotide (FAD) cofactor, oxidizes 1,2- and 1,6-NADPH NADP⁺, its biologically active form. The two electrons removed are then passed to molecular oxygen by the RNLS FAD cofactor forming hydrogen peroxide. This activity serves as an intracellular detoxification/metabolite repair function that alleviates inhibition of primary metabolism dehydrogenases by 1,2- and 1,6-NADPH molecules

Catalase. Catalase refers to any enzymes that breaks H₂O₂ down into H₂O and O₂ as shown in the following reaction.

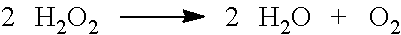

Catalases are found in almost all living organisms exposed to oxygen. Its primary function is to protects a cell or cells from oxidative damage associated with reactive oxygen species. In the present disclosure a catalase may be used to breakdown the H₂O₂ side product produced during the NAD detoxification reaction wherein NAD detoxifying enzymes oxidize 1,6-NADPH and 1,2-NADPH using O₂.

### NADPH Detoxification

The invention provides a NADPH detoxification device, for example as shown in FIG. 3. The NADPH detoxification device comprises an input line, a chamber comprising a NADPH detoxifying enzyme, and an output line wherein said input line is attached to said chamber at one end and said output line is attached to the end of the chamber opposite the input line. A cell-free reaction mixture comprising a mixture comprising 1,2-NADPH, 1,4-NADPH and/or 1,6-NADPH is passed through said NADPH detoxification device. An oxygen introducing element is attached to either the input line or the chamber. In some embodiments, the chamber further comprises a catalase enzyme that converts H₂O₂ side product produced from the detoxification reaction into H₂O and O₂. In some embodiments, a degassing element is attached to the output line to remove the oxygen not used in the detoxification reaction as well as O₂ produced by catalase.

In some embodiments, the NADPH detoxifying enzymes are in solution within the chamber of the NADPH detoxification device. In some embodiments, the NADPH detoxifying enzymes are immobilized by attachment to a substrate within the chamber of the NADPH detoxification device. In some embodiments, flavin mononucleotide (FMN) or flavin adenine dinucleotide (FAD) is added to the immobilized NADPH detoxification enzymes after they have been immobilized on the substrate. In some embodiments, a catalase may also be immobilized by attachment to the substrate.

When the NADPH detoxifying enzymes and optional catalases are attached to a substrate, they may be attached to any substrate sufficient to immobilize NADPH detoxifying enzymes and/or catalases within the chamber without interfering with the NADPH detoxification reaction in either affinity-based or non-affinity based methods. For instance, the solid support may include, without limitation, NiNTA beads, beads comprising antibodies or fragments thereof that specifically bind to the NADPH detoxifying enzymes or a tag found thereon, beads comprising protein A, streptavidin beads, cyanogen bromide-activated (CNBr) sepharose, glutaraldehyde crosslinking onto aminopropyl-containing silica, crosslinking and/or direct attachment with activated organic resins, etc.

If a resin is used as a solid support, the resin may be any size deemed necessary. For instance, the resin may be about 30 μm, about 40 μm, about 50 μm about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, or greater than 200 μm.

The NAD detoxification device comprises an effective amount of NADPH detoxifying enzymes to convert 1,2-NADPH and 1,-6-NADPH to NADP⁺ at a sufficient rate to maintain the desired productivity of a cell-free reaction.

The input line may be any object capable of carrying liquid without leakage. For instance, the input line may be, including without limitation, a tube, pipe, hollow bar, cylinder, hose etc. The output line may also be any object capable of carrying liquid without leakage. For instance, the output line may be, including without limitation, a tube, pipe, hollow bar, cylinder, hose etc. The input and output line may be the same object or they may be different objects.

The oxygen introducing element may be any element capable of introducing a sufficient amount of oxygen to support the detoxification reaction. For instance, the oxygen introducing element may be, including without limitation, a pump, a bubbler, etc.

The degassing element may be any element capable of removing most (i.e. >90%) of all of the oxygen within the cell-free reaction mixture (or cell-free recycling stream). For instance, the degassing element may be a pump, a vacuum, a membrane, etc.

### NAD Biosynthesis

In some embodiments, NAD biosynthesis enzymes are in solution within the chamber of the bioreactor. In some embodiments, NAD biosynthesis enzymes are immobilized by attachment to a substrate within the bioreactor chamber.

When NAD biosynthesis enzymes are attached to a substrate, they may be attached to any substrate sufficient to immobilize the enzymes within the chamber without interfering with the biosynthesis reaction in either affinity-based or non-affinity based methods. For instance, the solid support may include, without limitation, NiNTA beads, beads comprising antibodies or fragments thereof that specifically bind to the NAD biosynthesis enzymes or a tag found thereon, beads comprising protein A, streptavidin beads, cyanogen bromide-activated (CNBr) sepharose, glutaraldehyde crosslinking onto aminopropyl-containing silica, crosslinking and/or direct attachment with activated organic resins, etc.

If a resin is used as a solid support, the resin may be any size deemed necessary. For instance, the resin may be about 30 μm, about 40 μm, about 50 μm about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, or greater than 200 μm.

An effective amount of NAD biosynthesis enzymes are present to synthesis NAD at a sufficient rate to maintain the desired productivity of a cell-free reaction.

### Cofactor Biosynthesis Methods

In some embodiments, methods and compositions are provided for the in situ synthesis of a desired cofactor, e.g. NAD, in cell-free reactions mixtures in cell-free reactors to support the product of a cell-free reaction product.

For example, where biosynthesis of NAD is desired, enzymes (i) a ribokinase; (ii) a phosphoribosyl pyrophosphate (PRPP) synthetase (PRS; also known as ribose-phosphate diphosphokinase); (iii) nicotinamide phosphoribosyltransferase (NAMPT); and (iv) a nicotinamide-nucleotide adenylyltransferase (NMNAT) are provided in a cell-free reaction mixture comprising ribose, adenosine triphosphate (ATP) and nicotinamide. These enzymes may be referenced to as NAD biosynthesis enzymes. The NAD biosynthesis enzymes in the presence of ribose, ATP and nicotinamide lead the production of NAD⁺ where 1 ribose+1 nicotinamide+3 ATP results in the production of 1 NAD⁺+adenosine diphosphate (ADP)+adenosine monophosphate (AMP)+2 pyrophosphate (PPi). In some embodiments, bacterial cells are genetically modified to highly express one or more of the NAD biosynthesis enzymes.

In some embodiments, bacterial cells are modified to express high levels of two or more, three or more or all four NAD biosynthesis enzymes. When less than four of the NAD biosynthesis enzymes are highly expressed in the bacterial cell, the remainder of the NAD biosynthesis enzymes may be natively expressed by the bacterial cell. For instance, the bacterial cell may be modified to highly express the ribokinase but may natively express the PRS; NAMPT; and NMNAT.

The bacterial cells may be any bacterial cell of any species. For instance, the bacterial cell species may be Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The production of NAD⁺ is the result four distinct steps. First, ribokinase catalyzes the phosphoryl transfer from ATP to the O-5′ position on ribose thereby leading to the production of Ribose 5-phosphate+ADP. Second, PRS catalyzes the transfer of the terminal pyrophosphoryl group of ATP to ribose 5-phosphate to generate phosphoribosyl pyrophosphate (PRPP) and AMP. Third, NAMPT catalyzes the condensation of nicotinamide with phosphoribosyl pyrophosphate to yield nicotinamide mononucleotide (NMN)+pyrophosphate (PPi). Fourth, NMNAT catalyzes the conversion of NMN in the presence of ATP to NAD+ and PPi. Collectively, the four steps are summarized as 1 ribose+1 nicotinamide+3 ATP→1 NAD⁺+1 ADP+1 AMP+2PPi.

The NAD biosynthesis enzymes of the present disclosure can be any enzyme that performs the necessary steps to lead to the production of NAD+. For instance, the ribokinase may be any enzyme that falls under the enzyme commission number of EC 2.7.1.15. PRS may be any enzyme that falls under the enzyme commission number of EC 2.7.6.1. NAMPT may be any enzyme that falls under the enzyme commission number of EC 2.4.2.12. NMNAT may be any enzyme that falls under the enzyme commission number of EC 2.7.7.1. In some embodiments, the NAD biosynthesis enzymes have been modified to for enhanced catalytic activity.

In some embodiments, the ATP in the cell-free reactions is produced through oxidative phosphorylation. When ATP is generated by oxidative phosphorylation, oxygen (O₂) is added to the cell-free reaction mixture in the presence of cytochrome oxidase d thereby limiting the overall O₂ present within the cell-free reaction mixture. The concentration of cytochrome oxidase d is at a sufficient level to limit the amount of oxygen present in the cell-free reaction mixture such that the cell-free reaction mixture remains substantially anaerobic. The cytochrome oxidase d may be natively expressed by a bacterial cell or the bacterial cell may be modified to specifically express the cytochrome oxidase d at high levels. The cytochrome oxidase d may be any enzyme that catalyzes the oxidation of ubiquinol-8 in the presence of O₂ leading to the production of ubiquinone-8 in addition to H₂O. For instance, the cytochrome oxidase d may be any enzyme that falls under the enzyme commission number of EC 7.1.1.7.

In some embodiments, the concentration of ribose and nicotinamide is titrated in the cell-free reaction mixture to produce the desired concentration of NAD⁺. The desired concentration of NAD⁺ may be any NAD⁺ amount that specifically supports the production of a cell-free reaction mixture product. For instance, the desired concentration of NAD⁺ may be in the range from about 2 to about 5 mM. The desired concentration may be about 2 mM, about 3 mM, about 4 mM, about 5 mM, or more than 5 mM. In a preferred embodiment, the concentration of NAD⁺ is from about 2 mM to about 4 mM.

The cell-free reaction could be any reaction that produces a desired product that relies on the presence of NAD⁺. For instance, the cell-free reaction could be used to produce, including without limitation, commodity chemicals (i.e. succinate), proteins, small molecules, etc.

### Production Methods

High yield production of biomaterials of interest, e.g. carbon negative commodity chemicals, may be accomplished by providing a bioreactor for use with any, or any combination of the embodiments described herein, for example by inputting H₂ into a fluid in which NAD reducing pathway proteins are active, but use of detoxification, by production of cofactors in situ, etc.

Where used, the NAD reducing pathway proteins are produced in microbial cells. During cell culture it may be desirable to control the components of the growth medium of the cells in order to avoid exposure of the hydrogenase to conditions that affect activity, e.g. exposure to O₂ and the like. Generally the pathway components are produced in one or more different cells. For production purposes, a lysate of the cell is utilized. Cells are lysed by any convenient method that substantially maintains enzyme activity, e.g. sonication, French press, and the like as known in the art. The lysate may be fractionated, particulate matter spun out, etc., or may be used in the absence of additional processing steps. The cell lysate may be further combined with substrates, co-factors and such salts, buffers, etc. as are required for activity, and may be treated with iodoacetamide or a similar agent. Substrates will usually include H₂, glucose or another suitable sugar, a source of nicotinamide, and a source of ATP or adenine.

Enzymes for the synthetic and energy producing reactions are produced in microbial cells. For production purposes, a lysate of the cell is utilized. The lysate may be from a cell engineered to express levels of cofactor biosynthesis enzymes suitable for producing cofactors of interest. Alternatively, lysates can be supplemented with exogenous enzymes, which may be in solution or immobilized on a substrate. Cells are lysed by any convenient method that substantially maintains enzyme activity, e.g. sonication, French press, and the like as known in the art. The lysate may be fractionated, particulate matter spun out, etc., or may be used in the absence of additional processing steps. The cell lysate may be further combined with substrates, co-factors and such salts, buffers, etc. as are required for activity, and may be treated with iodoacetamide or a similar agent. Substrates may include O₂ and/or H₂, glucose or another suitable sugar, a source of nicotinamide, and a source of ATP or adenine.

Lysates of cells of different genetic backgrounds, e.g. previously altered or genetically engineered, or species, or that are prepared by different strategies can be mixed and simultaneously or sequentially used in a bioprocess with the cell lysate of the invention. The lysate can be free or immobilized, and can be reused or disposed at each stage of the process.

The reactions may utilize a large scale reactor, small scale, or may be multiplexed to perform a plurality of simultaneous syntheses. Continuous reactions will use a feed mechanism to introduce a flow of reagents, and may isolate the end-product as part of the process. Batch systems are also of interest, where additional reagents may be introduced over time to prolong the period of time for active synthesis or to limit the production of side products. A reactor may be run in any mode such as batch, extended batch, semi-batch, semi-continuous, fed-batch and continuous, and which will be selected in accordance with the application purpose.

The reactions may be of any volume, either in a small scale, usually at least about 1 ml and not more than about 15 ml, or in a scaled up reaction, where the reaction volume is at least about 15 ml, usually at least about 50 ml, more usually at least about 100 ml, and may be 500 ml, 1000 ml. The reaction volume may be greater than 1 L such as about 5 L, about 10 L, about 100 L, about 200 L, about 500 L, about 1000 L, about 5000 L, about 10000 L, about 20000 L, about 30000 L, about 40000 L, about 50000 L, about 100000 L, or greater. Reactions may be conducted at any scale.

Various salts and buffers may be included, where ionic species are typically optimized with regard to product production. When changing the concentration of a particular component of the reaction medium, that of another component may be changed accordingly. Also, the concentration levels of components in the reactor may be varied over time. The adjuster of the thiol/disulfide oxidation/reduction potential may be dithiothreitol, ascorbic acid, glutathione and/or their oxidized forms. Other adjusters of the general redox potential may also be used.

In a semi-continuous operation mode, the reactor may be operated in dialysis, diafiltration batch or fed-batch mode. A feed solution may be supplied to the reactor through the same membrane or a separate injection unit. The carbon negative commodity chemical, i.e. succinate, may be removed from the recycling stream. The reactor may be stirred internally or by proper agitation means, including by gas sparging or by directing the flow of a recycle inlet stream. The amount of carbon negative commodity chemical produced can be determined using chromatography and any instrument that measures the succinate concentration in the recycling stream, such as a high-performance liquid chromatographer (HPLC). The carbon negative commodity chemical can then be removed from the solution by any convenient means, as known in the art.

As explained above, the technology is deployable at large scale, can easily be implemented by building production facilities near bioethanol production facilities which exhaust CO₂ as a byproduct of bioethanol production. By providing CO₂ from the waste of external production processes, i.e. bioethanol production, the technology may achieve carbon negative production of commodity chemicals.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications that might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

1,6 NADH Oxidase Enzymes. The analytical conditions for resolution of the three NADH isomers has been determined. The 1,4-isomer is obtained from Sigma and the 1,6-isomer is isolated by preparative HPLC and confirmed by LC-MS and 1H-NMR (both available in the CBIS at RPI). The 1,2-isomer has low stability and is quantified in this work via mass balance closure. The 1,6- and 1,4-NADH standards are used to quantify rate and mass balances for NADH recycle and reactor detoxification. In addition to rhRen1 pyridoxamine-phosphate oxidases (PNPO) other PNPOs are prepared such as A. thaliana AtPPOX2, S. cerevisiae Ygr017w and N. punctiforme NpPPOX2.

The work has demonstrated the production of highly soluble rhRen1. Expression of PNPO from E. coli has been demonstrated. A third enzyme which is a PNPO with renalase-type activity from baker's yeast (PNPO-RP) has also been demonstrated. A hallmark of renalase and PNPO is the ability to oxidize 1,2- and 1,6-NADPH without oxidizing the productive 1,4-NADPH. Several PNPOs are known and their kinetic constants have been determined. The steady-state kinetic parameters of renalase, PNPO and PNPO—RP are confirmed under conditions relevant to our cell-free detoxification reaction via spectrophotometry and HPLC, as well as through a coupled assay that measures H₂O₂ production. Finally, soluble enzyme stability and deactivation kinetics (k_{inact}) are determined under operational conditions. The kinetic data allowed scale-up calculations and evaluation of enzyme immobilization effects.

### Production and Immobilization Processes

Renalase/PNPO immobilization allows for improved enzyme lifetime. Immobilization can be performed using standard approaches. Initial immobilization is performed using various affinity techniques, including Ni-NTA. If necessary, FMN is added post-immobilization to ensure that cofactor loss is not occurring. Immobilized enzyme kinetic performance is determined and compared to soluble enzyme kinetics. Non-affinity methods are then evaluated, including covalent attachment using CNBr-Sepharose, glutaraldehyde crosslinking onto aminopropyl-containing silica, and crosslinking and/or direct attachment with various commercial activated organic resins. These resins are in the size range of 50-150 μm to ensure acceptable flow properties within a packed-bed reactor (PBR). Covalently immobilized streptavidin is also evaluated to capture biotinylated renalase/PNPOs prepared using the avi-tag system. Immobilization efficiency (e.g., g renalase/g support) and enzyme kinetics are determined. The stability and reusability of the immobilized enzymes are evaluated, the latter by using the immobilized enzyme candidates in sequential reactions under identical reaction conditions.

Detoxification Reactor System. The initial target is an 8 mL PBR with a 1-min residence time for the five liter scale-up. Based on available kinetics of renalase and the PNPO-RP from S. cerevisiae, it is estimated a single pass conversion of 1,6-NADH to NAD⁺ of 48-72 μmol (6-9 mM). This estimate is tested by evaluating single-pass conversions as a function of 1,6-NADH concentration and flow rate. The stoichiometry is 1 mol O₂ per mol 1,6-NADH fed; however, some decoupling may occur. The total O₂ utilization is followed by measuring H₂O₂ formation. Because the H₂O₂ formed may be deleterious to the enzymes, catalase may also be immobilized to convert the H₂O₂ into H₂O and O₂. Operational stability of the PBR is determined as a function of time of operation, concentration of 1,6-NADH fed, flow rate and operational temperature, with the conversion to NAD⁺ as a key endpoint. Loss of activity over time may result from stripping FMN from the enzyme. This is addressed by periodic FMN replacement after taking the reactor offline.

PNPO improves protein production in cell-free reactions. A common and previously unexplained feature of modern cell-free protein synthesis (CFPS) protocols is a progressively declining production rate that cannot be recovered by augmenting reaction substrates and/or cofactors. Significant efforts to avoid the rate decreases has only provided modest benefits. The use of a natural enzyme, termed a renalase, has shown potential to provide metabolic benefits by detoxifying NADH and NADPH tautomers with reducing equivalents at the 1,6 and 1,2 positions instead of the normal 1,4 positions on the nicotinamide ring. This beneficial enzyme served as a 1,6- and 1,2-NADH oxidase using O₂ as the oxidant. Work showed that certain pyridoxamine-phosphate oxidases (also called PNPO's) could also deliver the same benefit. Such a PNPO was discovered in E. coli, a protein expressed by the PdxH gene.

CFPS protocols that rely upon oxidative phosphorylation to produce the ATP required to drive protein synthesis are typically used. This process is empowered by reducing equivalents delivered to the respiratory chain by NADH. It is also done in the presence of O₂ since the transfer of electrons from NADH to O₂ drives oxidative phosphorylation. It could therefore be reasoned that even if the 1,6- and 1,2-NADH tautomers arise during CFPS, the natural presence of the PdxH gene product in the cell extract and the supply of O₂ would detoxify the aberrant NADH forms.

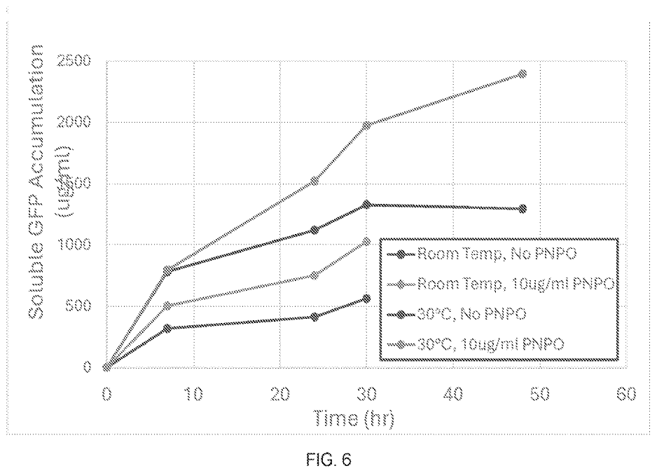

Surprisingly, data has shown (FIG. 6) that supplying additional E. coli PNPO enzyme significantly improves the accumulation of superfolder green fluorescent protein by more than 80% after incubation at both room temperature and at 30° C. This benefit becomes especially obvious after 24 hours of incubation.

TABLE 1

| | GFP Concentration | GFP Concentration |
|---|---|---|
| | (μg/ml) Control | (μg/ml) 10 μg/ml |
| Time Point | (No PNPO) Group | PNPO Group |
| T = 6-7 hr, room temp | 318 μg/ml | 503 μg/ml |
| T = 24 hr, room temp | 411 μg/ml | 751 μg/ml |
| T = 30 hr, room temp | 563 μg/ml | 1027 μg/ml |
| T = 6-7 hr, 30° C. | 781 μg/ml | 794 μg/ml |
| T = 24 hr, 30° C. | 1121 μg/ml | 1521 μg/ml |
| T = 30 hr, 30° C. | 1327 μg/ml | 1975 μg/ml |
| T = 48 hr, 30° C. | 1294 μg/ml | 2394 μg/ml |

### Example 2

Optimization by activating in situ synthesis of NAD or CoenzymeA (or both) Cell-free productivity increases from in situ cofactor synthesis. Cell extracts can be activated to conduct complex metabolic processes such as protein synthesis and the inexpensive production of commodity biochemicals and other bioproducts. However, producing the cell extracts typically lowers the concentrations of metabolic cofactors sufficiently to significantly lower the rates of numerous cofactor-dependent metabolic processes. While cofactors can be added to increase rates of product formation, this adds significant expense which could render many proposed commercial processes to be unprofitable.

This disclosure describes the controlled synthesis of metabolic cofactors from inexpensive substrates either using enzymes already present in a cell extract, added to the cell extract, or enzymes that have been produced by the cell extract. Since processes can work best when cofactors are provided at optimal concentrations, the extent of cofactor synthesis can be controlled by adding only the corresponding molar amounts of direct or indirect co-factor precursors. This example demonstrates protein yield increases obtained from in situ synthesis of NAD⁺ and CoenzymeA by adding ribose, nicotinamide, and/or pantothenate to an economical cell-free protein synthesis reaction.

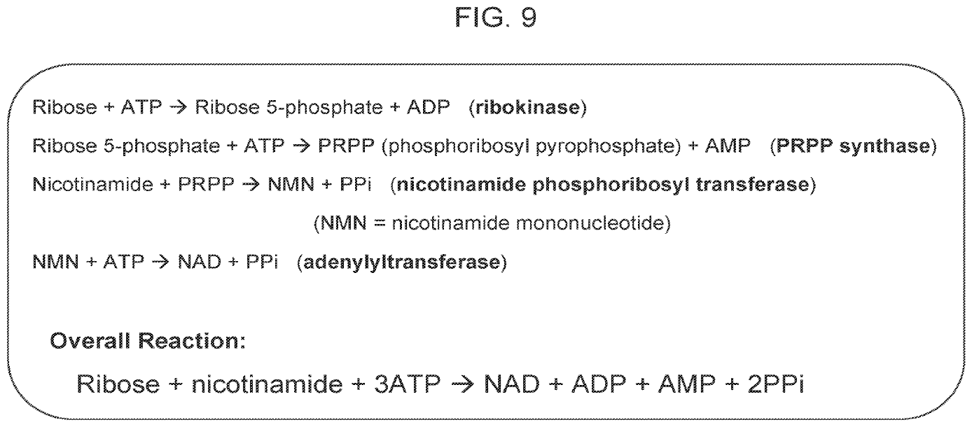

The initial economic analysis and the possibility of benefiting from up to 10 mM total NAD+ concentrations indicated that NAD+ purchase would impose unacceptable costs for succinate production and would also increase costs for protein production. The cost can be avoided by using a reaction scheme for in situ NAD biosynthesis. The production of NAD+ is the result of four distinct steps using reagents that are inexpensive or are provided by normally activated cell-free reactions (FIG. 9). First, ribokinase catalyzes the phosphorylation of ribose in presence of ATP and leads to the production of Ribose 5-phosphate+ADP. Second, PRS catalyzes the conversion of Ribose 5-phosphate in the presence of ATP to PRPP and AMP. Third, NAMPT catalyzes the conversion of nicotinamide in the presence of PRPP to nicotinamide mononucleotide (NMN)+PPi. Fourth, NMNAT catalyzes the conversion of NMN in the presence of ATP to NAD+ and PPi. Collectively, the four steps are summarized as 1 ribose+1 nicotinamide+3 ATP→1 NAD+1 ADP+1 AMP+2PPi.

The four enzymes required may be available in the cell extract or may be specifically added to the cell extract. Any of the individual four enzymes may be added to the extract or a combination thereof may be added. For instance, the extract may comprise ribokinase but may lack the PRS; the NAMPT; and NMNAT which then is supplemented to the cell-free reaction mixture or may be produced by the extract. Ribose and nicotinamide (niacin) are relatively inexpensive and are added as needed to maintain the targeted NAD+ concentration. Although ATP will generally not be available in excess during succinate production, small amounts can be produced by oxidative phosphorylation by oxygen infusion into the reactor. The induction of the cytochrome oxidase d during the cell extract fermentation will ensure that the dissolved oxygen concentration is maintained at a very low level since the cytochrome oxidase d has a very high affinity for oxygen. The oxygen tolerance of the mutated H₂ase will avoid damage to that enzyme. With this scheme, slight excesses of ribose and nicotinamide can be added to the reactor followed by slow O₂ infusion until the NAD⁺ concentration reaches the targeted level. Periodic production will be required because water coming in with the glucose feed for succinate production, for example, and being produced metabolically will increase the reaction fluid volume by about 1.5% per hour. To avoid overfilling or overflowing the reactor, this volume of recycle fluid will need to be removed and discarded resulting in NAD⁺ loss that requires replacement.

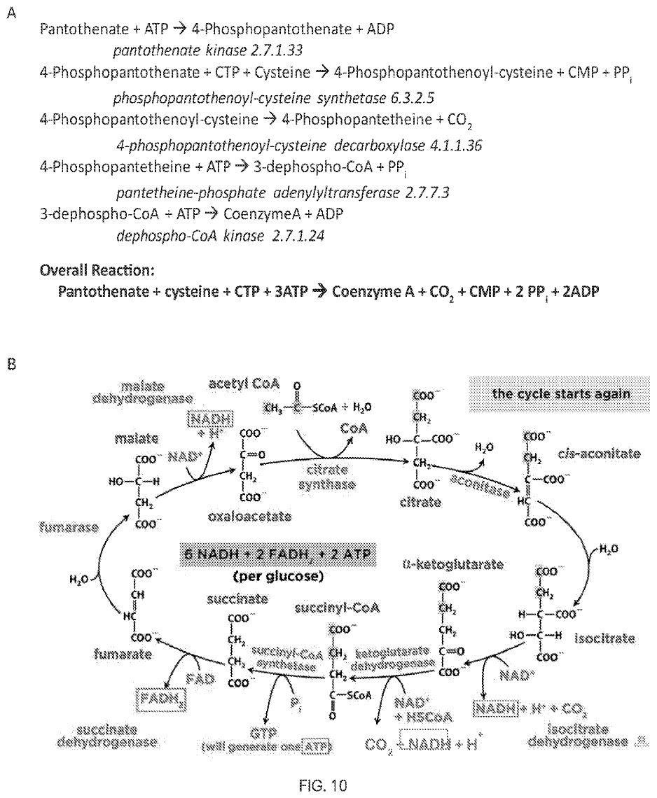

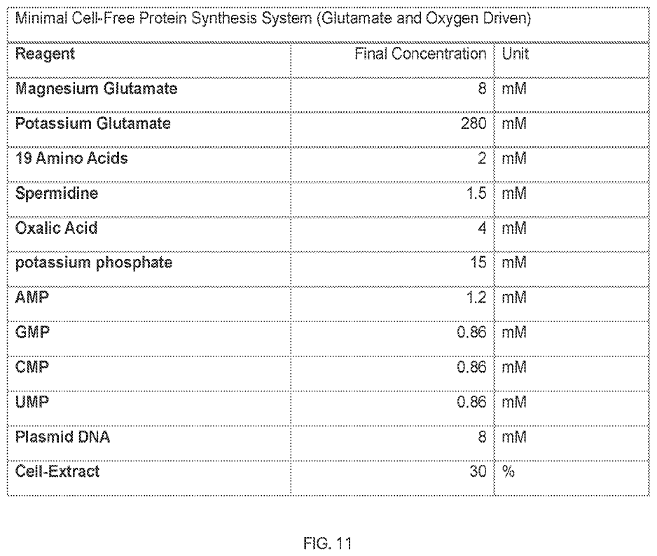

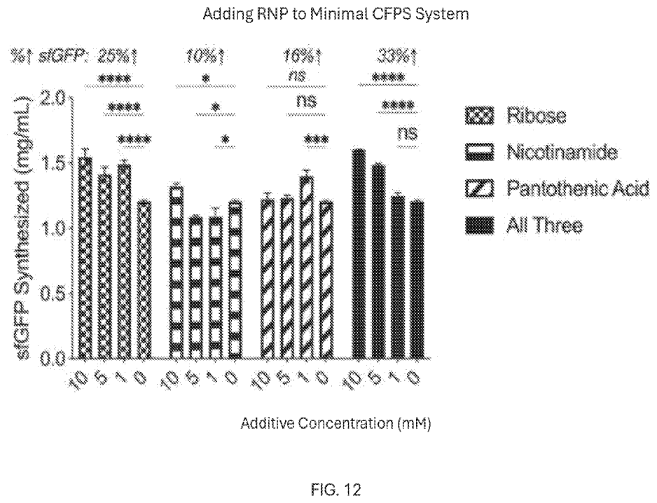

Similar advantages can be gained by in situ synthesis of CoenzymeA as well as NAD; for example, in reactions using large amounts of ATP generated by oxidative phosphorylation when glutamate is the primary source of reducing equivalents. These reducing equivalents (electrons) are harvested after glutamate is first converted to α-ketoglutarate and electrons are harvested by several of the molecular conversions catalyzed by the TCA (tricarboxylic acid) cycle enzymes (FIG. 10B. CoenzymeA is required to form the next intermediate in the cycle, succinylCoA. This reaction releases a CO₂ molecule and also harvests two electrons in the form of NADH. In this example, increased concentrations of both NAD and Coenzyme A could be advantageous. FIG. 10A shows the reactions and reactants required to produce CoenzymeA. FIG. 11 shows the reagents used to produce the protein, super folder green fluorescent protein (sfGFP). Note that CMP is provided as a substrate and is converted to CTP by kinases driven by the ATP produced by oxidative phosphorylation. Cysteine is provided as one of the 19 amino acids. FIG. 12 shows highly statistically significant 33% increase in sfGFP production when ribose, nicotinamide, and pantothenic acid (pantothenate) are all added. These reagents would not be expected to directly increase protein synthesis. They can only provide the observed benefit when they are converted into the metabolic cofactors produced by the synthetic sequences shown in FIG. 9 and FIG. 10.

### Example 3

FIG. 1 illustrates an overall integrated system for the production of desired biomaterials, e.g. carbon negative commodity chemicals. In this embodiment, cell extracts comprising (i) [FeFe] hydrogenase; (ii) ferredoxin; and (iii) ferredoxin-NADP-reductase (FNR) or ferredoxin-NAD+-reductase (FN+R) are added to a cell free reactor. In addition to the cell extracts, glucose, CO₂, and highly pressurized H₂ are injected into the cell free reactor. The cell free reactor solution is pumped into a recirculation stream to separate products from reactants and proteins present in the fluid.

The H₂ necessary for the synthesis of reducing equivalents of NAD/P(+) and the production of carbon negative commodity chemicals may be injected into the cell free reactor using any means, e.g. by direct injection, or by injecting into a pressurized stream of the recirculation fluid.

### Example 4

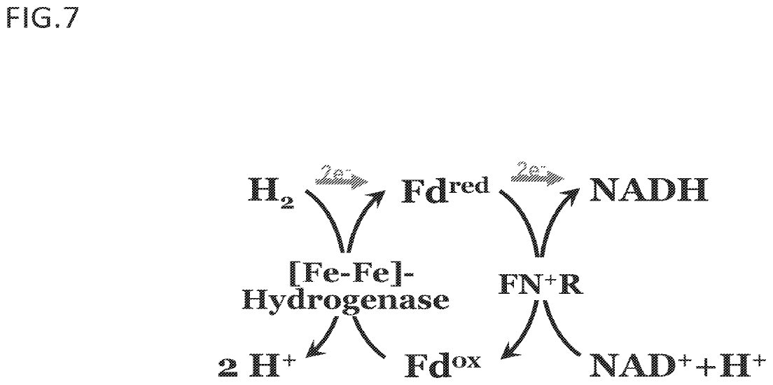

Optimize Hydrogen to NADH Pathway. FIG. 7 shows the electron transfer reactions that lead to the reduction of NAD⁺ to form NADH. The [FeFe]H₂ase, Cpl, shows very rapid electron transfer when using the Clostridium pasteurianum ferredoxin (CpFd) protein that has evolved to dock effectively at the Cpl electron transfer surface location. Key FN⁺R that find use in the present disclosure display abilities to readily accept electrons from CpFd and to efficiently deliver them to NAD⁺. FN⁺Rs are evaluated in the H₂ to NADH pathway while using pyruvate and lactate hydrogenase to avoid NADH accumulation. Lactate accumulation rate indicates FN⁺R activity. FN⁺R that display the desired abilities are administered at varying CpFd to FN⁺R ratios to identify the best pathway composition.

A succinate productivity of 10 g/L-hr requires about 90 mmoles of NAD⁺ reduction/L-hr since 1 extra NADH is needed for each succinate produced. Considering that FNRs often catalyze turnover rates of 50/sec and Cpl catalyzes several hundred reactions per second, the required concentrations are low (for example about 5 UM for FNR and about 0.5 UM for the hydrogenase).

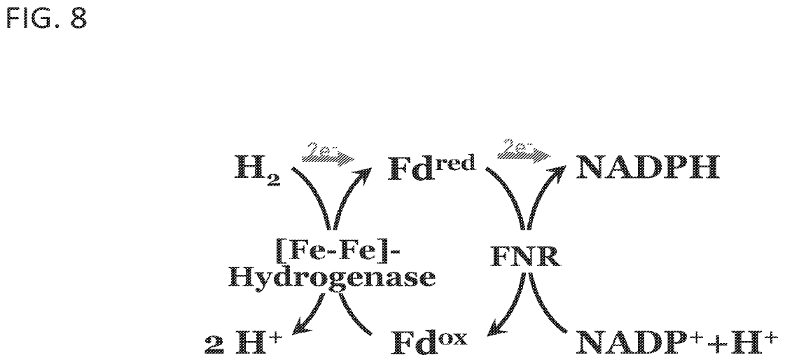

Providing metabolic reducing equivalents from electricity. Because of the problems with direct NAD+ reduction, electrolyzers are in use to split water to provide H₂ and deliver reducing equivalents to the bioreactor. Another example is shown in FIG. 8 for the reduction of NADP⁺ to NADPH.

Effective Hydrogen Mass Transfer. Since hydrogen has low solubility in aqueous solutions, hydrogen mass transfer from the gaseous phase to the liquid phase is considered. Initial process analysis indicates that the 10 g/L-hr succinate productivity can be sustained by a transfer rate of about 1.4 mmoles/L-min. Electrolyzers are used to provide H₂ as a pressurized gas. A positive displacement pump is placed in the recycle loop to increase the filtrate pressure. H₂ is introduced into the recycle fluid through nozzles to supersaturate the fluid with H₂. The recycle stream will then be returned into the large cell-free reactor through nozzles that assist in providing bulk mixing while also maintaining the elevated pressure in the H₂ injection section of the recycle loop. The H₂ emerges from the injected fluid as small bubbles of pure H₂ to provide excellent mass transfer.

### REFERENCES

1. Jantama K, Zhang X, Moore J C, Shanmugam K T, Svoronos S A, Ingram L O. (2008) Eliminating side products and increasing succinate yields in engineered strains of Escherichia coli. Biotechnology and Bioengineering 101 (5): 881-93. DOI 10.1002/bit22005
2. Jewett M C, Swartz J R. (2004) Substrate replenishment extends protein synthesis with an in vitro translation system designed to mimic the cytoplasm. Biotechnol Bioeng. 87 (4): 465-72. doi: 10.1002/bit.20139.
3. Jewett M C, Calhoun K A, Voloshin A, Wuu J J, Swartz J R. (2008) An integrated cell-free metabolic platform for protein production and synthetic biology. Mol Syst Biol. 4:220. doi: 10.1038/msb.2008.57.
4. Swartz J R. (2011) Transforming biochemical engineering with cell-free biology. AIChE Journal 58 (1): 5-13. DOI 10.1002/aic.13701
5. Armiger, T. J., Armiger, W. B., Dodds, D. R. 2012. Electrochemical Bioreactor Module and Methods of Using the Same. European #2,893,587; China #CN104871357; Hong Kong #HK1,214,412 September 9. (pending U S, Canada & Brazil)
6. Armiger, W. B., Dodds, D. R . . . 2014. Improved Electrochemical Bioreactor Module and Use Thereof. United States of America patent W P Appl. WO2016070168A1. November 2. (pending U S, European, China, Canada, Hong Kong & Brazil)
7. Mädje, K., Schmölzer, K., Nidetzky, B. & Kratzer, R. Host cell and expression engineering for development of an E. coli ketoreductase catalyst: Enhancement of formate dehydrogenase activity for regeneration of NADH. Microb. Cell Fact. 11, 7 (2012).
8. Chenault, H. K. & Whitesides, G. M. Regeneration of nicotinamide cofactors for use in organic synthesis. Appl. Biochem. Biotechnol. 14, 147-197 (1987).
9. Shin, I. H., Jeon, S. J., Park, H. S. & Park, D. H. Catalytic oxidoreduction of pyruvate/lactate and acetaldehyde/ethanol coupled to electrochemical oxidoreduction of NAD+/NADH. J. Microbiol. Biotechnol. 14, 540-546 (2004).
10. Xu, J. et al. Renalase is a novel, soluble monoamine oxidase that regulates cardiac function and blood pressure. J. Clin. Invest. 115, 1275-1280 (2005).
11. Hoag, M. R., Roman, J., Beaupre, B. A., Silvaggi, N. R. & Moran, G. R. Bacterial Renalase: Structure and Kinetics of an Enzyme with 2- and 6-Dihydro-β-NADP Oxidase Activity from Pseudomonas phaseolicola. Biochemistry 54, 3791-3802 (2015).
12. Beaupre, B. A., Hoag, M. R., Roman, J., Försterling, F. H. & Moran, G. R. Metabolic function for human renalase: oxidation of isomeric forms of β-NADPH that are inhibitory to primary metabolism. Biochemistry 54, 795-806 (2015).
13. Marbaix, A. Y. et al. Pyridoxamine-phosphate oxidases and pyridoxamine-phosphate oxidase-related proteins catalyze the oxidation of 6-NADPH to NADP+. Biochem. J. 476, 3033-3052 (2019).
14. Morrison, C. S., Armiger, W. B., Dodds, D. R. 2016. Improved Method for Using Electrochemical Bioreactor Module with Recovery of Cofactor. US patent Allowed, Appl. WO2017160793. March 14. (pending European, China, Canada, Hong Kong & Brazil)
15. Morrison, C. S., Armiger, W. B., Dodds, D. R., Dordick, J. S. & Koffas, M. A. G. Improved strategies for electrochemical 1,4-NADPH<inf>2</inf> regeneration: A new era of bioreactors for industrial biocatalysis. Biotechnol. Adv. 36, (2018)
16. Smith P R, Bingham A S., Swartz J. (2012) Generation of hydrogen from NADPH using an [FeFe] hydrogenase. International journal of hydrogen energy. 37(3):2977-2983. doi: 10.1016/j.ijhydene.2011.03.172.
17. Lu F, Smith P, Mehta K, Swartz J. (2015) Development of a synthetic pathway to convert glucose to hydrogen using cell free extracts. International journal of hydrogen energy 40:9113-24. doi: 10.1016/j.ijhydene.2015.05.12.
18. Kuchenreuther J M, Grady-Smith C S, Bingham A S, George S J, Cramer S P, Swartz J R. (2010) High-yield expression of heterologous [FeFe] hydrogenases in Escherichia coli. PLoS One. 5(11):e15491. doi: 10.1371/journal.pone.0015491
19. Koo J, Swartz J R. (2018) System analysis and improved [FeFe] hydrogenase O₂ tolerance suggest feasibility for photosynthetic H₂ production. Metab Eng. 49:21-27. doi: 10.1016/j.ymben.2018.04.024.
20. Keiler, K. C. (n.d.). Role of a Peptide Tagging System in Degradation of Proteins Synthesized from Damaged Messenger RNA Peptides for PPI inhibition View project. https://doi.org/10.1126/science.271.5251.990
21. Keiler, K. C., & Sauer, R. T. (1996). Sequence Determinants of C-terminal Protein Recognition by the Tsp Protease*.
22. Gottesman, S., Roche, E., Zhou, Y. N., & Sauer, R. T. (1998). The ClpXP and ClpAP proteases degrade proteins with carboxy-terminal peptide tails added by the SsrA-tagging system. Genes and Development, 12(9), 1338-1347. https://doi.org/10.1101/gad.12.9.1338
23. Gur, E., & Sauer, R. T. (2008). Evolution of the ssrA degradation tag in Mycoplasma: Specificity switch to a different protease. Proceedings of the National Academy of Sciences of the United States of America, 105 (42), 16113-16118. https://doi.org/10.1073/pnas.0808802105
24. McGinness, K. E., Baker, T. A., & Sauer, R. T. (2006). Engineering Controllable Protein Degradation. Molecular Cell, 22(5), 701-707. https://doi.org/10.1016/j.molcel.2006.04.027
25. Zawada J, Swartz J R. (2005) Maintaining rapid growth in moderate-density Escherichia coli fermentations. Biotechnology and Bioengineering 89 (4): 407-15. DOI: 10.1002/bit.20369
26. Kuchenreuther J M, Myers W K, Stich T A, George S J, Nejatyjahromy Y, Swartz J R, Britt R D. (2013) A radical intermediate in tyrosine scission to the C O and C N-ligands of FeFe hydrogenase. Science. 342 (6157): 472-5. doi: 10.1126/science. 1241859.
27. Kuchenreuther J M, Myers W K, Suess D L, Stich T A, Pelmenschikov V, Shiigi S A, Cramer S P, Swartz J R, Britt R D, George S J. (2014) The HydG enzyme generates an Fe(CO)2(CN) synthon in a assembly of the FeFe hydrogenase H-cluster. Science. 2014 Jan. 24; 343(6169):424-7. doi: 10.1126/science.1246572.
28. Suess D L, Bürstel I, De La Paz L, Kuchenreuther J M, Pham C C, Cramer S P, Swartz J R, Britt R D. (2015) Cysteine as a ligand platform in the biosynthesis of the FeFe hydrogenase H cluster. Proc Natl Acad Sci USA. 112(37):11455-60. doi: 10.1073/pnas.1508440112.
29. Morrison, C. S. et al. Improved soluble expression and use of recombinant human renalase. PLoS One 15, e0242109 (2020).
30. Kim, D. et al. Modular Assembly of Unique Chimeric Lytic Enzymes on a Protein Scaffold Possessing Anti-Staphylococcal Activity. Biomacromolecules 20, 4035-4043 (2019).
31. Nakimbugwe, D. et al. Cell wall substrate specificity of six different lysozymes and lysozyme inhibitory activity of bacterial extracts. FEMS Microbiol. Lett. 259, 41-46 (2006).
32. Briers, Y. et al. Engineered Endolysin-Based “Artilysins” To Combat Multidrug—. MBio 5, e01379-14 (2014).
33. Doud, D. F. R., Jain, A., Ahsan, S. S., Erickson, D. & Angenent, L. T. In situ UV disinfection of a waveguide-based photobioreactor. Environ. Sci. Technol. 48, 11521-11526 (2014).
34. Swartz, J R, “Recombinant cellular lysate system for producing a product of interest” U.S. Pat. No. 10,036,001. Issued Jul. 31, 2018
35. Swartz, J R, “Methods for control of flux in metabolic pathways through enzyme relocation” U.S. Pat. No. 10,006,062. Issued Jun. 26, 2018
36. Smith P R and J R Swartz, “Hydrogenase fusion protein for improved hydrogen production” U.S. Pat. No. 9,133,486 Issued Sep. 15, 2015.
36. Swartz J R, “Methods for control of flux in metabolic pathways through enzyme relocation” U.S. Pat. No. 8,956,833 Issued Feb. 17, 2015.
37. Swartz J R, “Methods for control of flux in metabolic pathways through protease manipulation” U.S. Pat. No. 8,916,358 Issued Dec. 23, 2014.

What is claimed is:

1. A method of increasing productivity of a cell-free reaction mix for a reaction requiring a cofactor of interest for synthesis of a desired biomaterial, the method comprising:
manipulating cofactor activity in the reaction mixture.

2. The method of claim 1, wherein the cofactor of interest is nicotinamide adenine dinucleotide (NAD).

3. The method of claim 1 or claim 2, wherein the cofactor of interest is coenzyme A.

4. The method of any of claims 1-3, wherein the cell-free reaction is in a bioreactor.

5. The method of any of claims 1-4, wherein the method increases the production of the desired biomaterial by at least about 20% relative to the production of the desired biomaterial in the absence of the cofactor activity manipulation.

6. The method of any of claims 1-5, wherein the reaction mixture comprises an E. coli cell lysate.

7. The method of any of claims 1-6, wherein the reaction mixture is used to produce a commodity chemical, optionally succinate.

8. The method of any of the preceding claims, comprising:
providing cofactor biosynthesis enzymes and cofactor biosynthesis substrates in the reaction mix in an amount effective to produce cofactor(s) of interest.

9. The method of any of the previous claims, wherein the desired biomaterial is concomitantly synthesized with the cofactor of interest.

10. The method of any of claims 8-9, wherein one or more of cofactor biosynthesis enzymes are synthesized by a microbial cell genetically modified to express the cofactor biosynthesis enzyme, optionally wherein the genetically modified microbial cell is lysed to provide the microbial cell lysate of the reaction mixture.

11. The method of any of claims 8-10, wherein one or more isolated cofactor biosynthesis enzymes is added to the reaction mixture.

12. The method of any of claims 8-11, wherein one or more cofactor biosynthesis enzymes are soluble in the reaction mixture.

13. The method of any of claims 8-11, wherein one or more cofactor biosynthesis enzymes are immobilized on a substrate in the bioreactor.

14. The method of any of claims 8-13, wherein the cofactor biosynthesis enzymes comprise one or more of (i) ribokinase; (ii) phosphoribosyl pyrophosphate (PRPP) synthetase;
(iii) nicotinamide phosphoribosyltransferase (NAMPT); and (iv) nicotinamide-nucleotide adenylyltransferase (NMNAT).

15. The method of any of claims 8-13, wherein the cofactor biosynthesis enzymes comprise each of (i) ribokinase; (ii) phosphoribosyl pyrophosphate (PRPP) synthetase; (iii) nicotinamide phosphoribosyltransferase (NAMPT); and (iv) nicotinamide-nucleotide adenylyltransferase (NMNAT).

16. The method of any of claims 8-15, wherein the cofactor biosynthesis substrates comprise nicotinamide; ribose; and adenosine triphosphate (ATP).

17. The method of claim 16, wherein the source of nicotinamide is in the form of niacin.

18. The method of any of claims 8-17, wherein the cofactor biosynthesis enzymes comprise one or more of (i) pantothenate kinase; (ii) phosphopantetheinyl-cysteine synthetase; (iii) 4-phosphopantetheinyl-cysteine decarboxylase; (iv) pantetheine-phosphate adenylyltransferase and (v) dephospho-CoA.

19. The method of any of claims 8-17, wherein the cofactor biosynthesis enzymes comprise each of (i) pantothenate kinase; (ii) phosphopantetheinyl-cysteine synthetase; (iii) 4-phosphopantetheinyl-cysteine decarboxylase; (iv) pantetheine-phosphate adenylyltransferase and (v) dephospho-CoA.

20. The method of claim 18 or 19, wherein the cofactor biosynthesis substrates comprise pantothenate, cysteine, CTP and ATP.

21. The method of any of the preceding claims, wherein the reaction mixture further comprises cytochrome oxidase d.

22. The method of any of the preceding claims, wherein the reaction mixture further comprises oxygen (O₂).

23. The method of any of claims 8-19, wherein the concentration of ribose and nicotinamide is adjusted to maintain a NAD concentration from about 2 mM to about 10 mM in the reaction mixture.

24. The method of any of the preceding claims, comprising:
contacting the cell-free reaction mixture one or more NADPH detoxifying enzymes in the presence of O₂, wherein 1,6-NADPH, 1,2-NADPH or a mixture thereof are converted to NADP⁺.

25. The method of claim 24, wherein the NADPH detoxifying enzymes are immobilized on a substrate, optionally a resin.

26. The method of claim 24 or 25, wherein the NADPH detoxifying enzymes are provided in a recirculation stream operably attached to the bioreactor chamber.

27. The method of any of claims 24-26, wherein the NADPH detoxifying enzyme is selected from Saccharomyces cerevisiae Pdx3, Arabidopsis thaliana PPOX1, Escherichia coli PdxH, Mus musculus PNPO; A. thaliana PPOX1, Nostoc punctiforme NpPPOX2, S. cerevisiae Ygr017w, Homo sapiens Renalase, modified H. sapiens Renalase m5hRen1 and modified H. sapiens Renalase m6hRen1.

28. The method of any of claims 24-27, wherein the NADPH detoxifying enzyme comprises a tag for immobilization to a substrate selected from polyhistidine or a variant thereof, FLAG, glutathione S-transferase (GST), green florescent protein (GFP) or a variant thereof, biotin, hemagglutinin (HA), maltose binding protein (MBP), Myc, small ubiquitin like modifier (SUMO), and ubiquitin.

29. The method of any of claims 24-28, wherein the NADPH detoxifying enzymes are linked to a substrate through click chemistry or through a bi-functional linker.

30. The method of any of the preceding claims, comprising:
synthesizing reducing equivalents from gaseous H₂, the method comprising:
combining in the cell-free bioreactor enzymes (i) active [FeFe] hydrogenase; (ii) ferredoxin; and (iii) ferredoxin-NAD⁺-reductase (FN⁺R), NAD⁺ or NADP⁺, and a source of supersaturated H₂ and
incubating the reaction for a period of time sufficient to produce NADH or NADPH.

31. The method of claim 30, wherein at least 90 mmoles of NAD+ is reduced/L-hr.

32. The method of claim 30 or 31, wherein one or more of the cell-free bioreactor enzymes are synthesized by a genetically modified microbial cell.

33. The method of claim 32, wherein the genetically modified microbial cell is lysed to provide the microbial cell lysate of the reaction mixture.

34. The method of any of claims 30-33, wherein the [FeFe] hydrogenase and ferredoxin are Clostridium pasteurianum enzymes.

35. The method of any of claims 30-34, wherein the active [FeFe] hydrogenase is an oxygen tolerant hydrogenase.

36. The method of any of claims 30-35, further comprising adding to the reaction mix an exogenous source of carbohydrates.

37. A reaction mixture for use in the method of any of the preceding claims.

38. A bioreactor synthetic system comprising
(i) a main bioreactor chamber comprising cell-free aqueous bioreactor fluid, in which H₂ is present at sufficiently high partial pressure to force transfer from the gas phase into the aqueous phase.

39. The bioreactor synthetic system of claim 38, wherein (ii) an H₂ input element into the main chamber feeds a supersaturated aqueous H₂ solution into the bioreactor fluid.

40. The bioreactor synthetic system of claim 39, comprising (iii) an output element from the main chamber into which is fed a stream of bioreactor aqueous fluid, or a filtrate obtained from aqueous reactor fluid, (iv) an H₂ injection element feeding gaseous H₂ into the output element at a pressure sufficient for the gaseous H₂ to be fully dissolved within the stream creating a supersaturated H₂ stream, and (v) an operable connection from the pressurized H₂ stream to the (ii) H₂ input stream.

41. The bioreactor synthetic system of claim 40, wherein the (iv) H₂ injection element is a nozzle or back pressure regulator device.

42. The bioreactor synthetic system of claim 38, wherein (vi) an H₂ input element directly injects gaseous H₂ at high pressure into the main bioreactor chamber.

43. The bioreactor synthetic system of any of claims 38-42, comprising a mixing element in the (i) main chamber to distribute the supersaturated aqueous H₂ solution in the bioreactor fluid.

44. The bioreactor synthetic system of any of claims 38-43, wherein the (i) main chamber is substantially anaerobic.

45. The bioreactor synthetic system of any of claims 38-44, wherein the gaseous H₂ is generated by an electrolyzer using electricity to generate H₂ and O₂ from H₂O, where the O₂ is expelled from the anodic chamber and the H₂ is expelled from the cathodic chamber.

46. The bioreactor synthetic system of any of claims 38-45, wherein the H₂ has a mass transfer rate in the bioreactor fluid of about 2 mmol L⁻¹ min⁻¹.

47. The bioreactor of any of claims 38-46, wherein the bioreactor fluid comprises microbial cell lysates or enzymes derived therefrom comprising sufficient NAD reducing pathway enzymes to generate NADH or NADPH from NAD⁺ or NADP⁺ and H₂.

48. The bioreactor of claim 47, wherein the NAD⁺ reducing pathway enzymes comprise (i) active [FeFe] hydrogenase; (ii) ferredoxin; and (iii) ferredoxin-NADP-reductase (FNR) or ferredoxin-NAD⁺-reductase (FN⁺R).

49. An NADPH detoxification device, the device comprising:
a) an input line,
b) a chamber comprising a NADPH detoxifying enzyme, and
c) an output line;
wherein said input line is attached to said chamber at one end and said output line is attached to the end of the chamber opposite of the input line.

50. The device of claim 49, wherein the NADPH detoxifying enzymes are immobilized on a substrate.

51. The device of any of claims 49-50, wherein an oxygen introducing element is attached to the input line or the chamber.

52. The device of any of claims 49-51, wherein a degassing element is attached to the output line.

53. The device of any of claims 49-52, wherein the NADPH detoxifying enzyme is selected from Saccharomyces cerevisiae Pdx3, Arabidopsis thaliana PPOX1, Escherichia coli PdxH, Mus musculus PNPO; A. thaliana PPOX1, Nostoc punctiforme NpPPOX2, S. cerevisiae Ygr017w, Homo sapiens Renalase, modified H. sapiens Renalase m5hRen1 and modified H. sapiens Renalase m6hRen1.

54. The device of any of claims 49-53, wherein the NADPH detoxifying enzyme is immobilized to a substrate selected from NiNTA beads, beads comprising antibodies or fragments thereof that specifically bind to the NADPH detoxifying enzymes or a tag found thereon, beads comprising protein A, streptavidin beads, cyanogen bromide-activated (CNBr) sepharose, glutaraldehyde crosslinked to aminopropyl-containing silica, and activated organic resins.

55. The device of any of claims 49-54, further comprising a catalase, optionally attached to a substrate.

56. The device of any of claims 49-55, wherein the NADPH detoxifying enzymes and/or catalase are linked to a substrate through click chemistry or through a bi-functional linker.

57. The device of any of claims 49-56, further comprising flavin mononucleotide (FMN) or flavin adenine dinucleotide (FAD).

58. The device of any of claims 49-57, operably connected to a bioreactor.
